(19) United States
(12) Patent Application Publication
(10) Pub. No.: US 2019/0054070 A1
(43) Pub. Date: Feb. 21, 2019

(54) Methods and Compositions for Regenerative Synaptogenesis

(71) Applicant: Daniel L. Alkon, Bethesda, MD (US)

(72) Inventor: Daniel L. Alkon, Bethesda, MD (US)

(21) Appl. No.: 15/923,277

(22) Filed: Mar. 16, 2018

Related U.S. Application Data
(60) Provisional application No. 62/473,116, filed on Mar. 17, 2017.

(51) Int. Cl. A61K 31/4155; A61P 25/00; A61K 31/366; A61K 31/454; A61K 31/4245; A61K 31/7076; A61K 31/185

(52) U.S. Cl. CPC A61K 31/4155; A61P 25/00; A61K 31/366; A61K 31/185; A61K 31/4245; A61K 31/7076; A61K 31/454

(57) ABSTRACT

Provided herein are uses of pharmaceutically effective amounts of an inhibitor of an arginine methyltransferase alone or in combination with one or more bryostatins to promote regenerative synaptogenesis. Also provided are methods for promoting regenerative synaptogenesis in a patient by administering a pharmaceutically effective amount of an inhibitor of an arginine methyltransferase alone or in combination with a PKC activator to the patient. Likewise, methods are also provided are methods for promoting dendritic maturation in a patient by administering a pharmaceutically effective amount of an inhibitor of an arginine methyltransferase alone or in combination with a PKC activator to the patient, wherein the administration promotes synaptic formation in the hippocampus.

### RELATED APPLICATIONS

This application claims priority to and the benefit of U.S. Provisional Patent Application No. 62/473,116, filed Mar. 17, 2017, and is incorporated herein by reference in its entirety.

### FIELD OF THE INVENTION

This invention relates generally to the field of synaptogenesis.

### BACKGROUND OF THE INVENTION

An improved understanding of the molecular mechanisms in synapse formation provides insight into both learning and memory as well as the etiology of neurodegenerative disorders.

There exists a need for additional methods and composition suitable for promoting regenerative synaptogenesis.

### SUMMARY OF THE INVENTION

Provided herein are uses of a pharmaceutically effective amount of bryostatin 1 in combination with a pharmaceutically effective amount of an inhibitor of an arginine methyl transferase to promote regenerative synaptogenesis.

Also provided are uses of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof in combination with a pharmaceutically effective amount of an inhibitor of an arginine methyl transferase to promote regenerative synaptogenesis.

Likewise, additionally provided are uses of a pharmaceutically effective amount of an inhibitor of an arginine methyl transferase to promote regenerative synaptogenesis.

In any of the uses disclosed herein, the inhibitor of an arginine methyl transferase is selected from the group consisting of S-(5′-Adenosyl)-L-homocysteine; 7,7′-(carbonylbis(azanediyl))bis(4-oxidonaphthalene-2-sulfonate), sodium salt (AMI-1); 5′-Deoxy-5′(methylthio)adenosine;

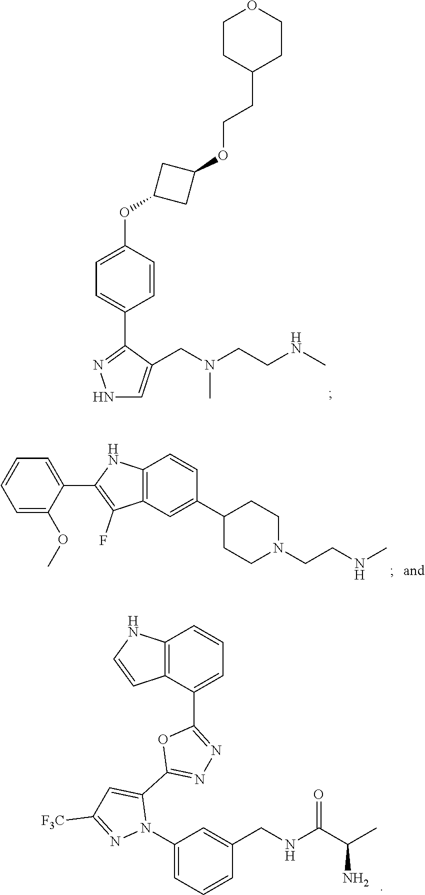 [SMILES: CNCCN(C)Cc1c[nH]nc1-c1ccc(O[C@H]2C[C@H](OCCC3CCOCC3)C2)cc1 ; CNCCN1CCC(c2ccc3[nH]c(-c4ccccc4OC)c(F)c3c2)CC1 ; C[C@@H](N)C(=O)NCc1cccc(-n2nc(C(F)(F)F)cc2-c2nnc(-c3cccc4[nH]ccc34)o2)c1]

In any of these uses, the pharmaceutically effective amount is from about 0.0000001 mg/kg to about 250 mg/kg per dose (i.e., from about 0.00001 mg/kg to about 5.0 mg/kg per dose).

In some embodiments, the pharmaceutically effective amount of bryostatin 1 is provided in a dose from 0.01-25 μg/m² intravenously.

In any of the uses disclosed herein, the arginine methyl transferase is Coactivator-associated Arginine Methyltransferase 1 (CARM1).

Also provided are methods for promoting regenerative synaptogenesis in a patient by administering a pharmaceutically effective amount of a PKC activator in combination with a pharmaceutically effective amount of an inhibitor of an arginine methyltransferase to said patient, wherein the patient is suffering from a disease, disorder, or condition characterized by inadequate or deficient synaptogenesis. Likewise, also provided are methods for promoting regenerative synaptogenesis in a patient by administering a pharmaceutically effective amount of an inhibitor of an arginine methyltransferase to said patient, wherein the patient is suffering from a disease, disorder, or condition characterized by inadequate or deficient synaptogenesis.

Additionally provided are methods for promoting dendritic maturation in a patient by administering a pharmaceutically effective amount of a PKC activator in combination with an inhibitor of an arginine methyltransferase to said patient, wherein the administration promotes synaptic formation in the hippocampus as well as methods for promoting dendritic maturation in a patient by administering a pharmaceutically effective amount of an inhibitor of an arginine methyltransferase to said patient, wherein the administration promotes synaptic formation in the hippocampus.

In any of these methods, the PKC activator activates the PKC ε isozyme and/or the PKC α isozyme.

In some embodiments, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

Other PKC activators that can be used in any of the methods disclosed herein, include, but are not limited to, one or more of a polyunsaturated fatty acid, a potassium channel activator, a neristatin, phorbol-12-myristate-13-acetate (PMA), okadaic acid, 1α,25-dihydroxyvitamin D3, 12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino)benzolactam (α-amyloid precursor protein modulator), cis-9-octadecenoic acid (oleic acid), ingenol 3-angelate, resiniferatoxin, L-α-Phosphatidyl-D-myo-inositol-4,5-bisphosphate, triammonium salt (PIP2), phorbol-12, 13-dibutyrate, 8(S-hydroxy-(5Z, 9E, 11Z, 14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin and its analogs, resiniferonol 9,13,14-ortho-phenylacetate, C-8 ceramide, 1,6-bis(Cyclohexyloximinocarbonylamino) hexane; 1,6-Di(O-(carbamoyl)cyclohexanone oxime) hexane (RHC-80267), (+/−)-1-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, or arachidonic acid-d8. For example, the potassium channel activator can be a diazoxide and/or the neristatin is neristatin 1.

In any of these methods, the arginine methyltransferase is Coactivator-associated Arginine Methyltransferase 1 (CARM1).

In any of the methods disclosed herein, the PKC activator and/or the arginine methyltransferase inhibitor are administered orally, intraperitoneally, subcutaneously, intranasally, buccally, trans-dermally, intramuscularly, intrarectally, intravenously, by inhalation, or any combination thereof.

Determination of the pharmaceutically effective amounts of the PKC activator and/or the inhibitor of the arginine methyltransferase is within the routine level of skill in the art. For example, the pharmaceutically effective amount of the PKC activator and/or the inhibitor of the arginine methyltransferase is from about 0.0000001 mg/kg to about 250 mg/kg per dose.

In one embodiment, the pharmaceutically effective amount of bryostatin 1 is from about 0.00001 mg/kg to about 5.0 mg/kg per dose. In another embodiment, the pharmaceutically effective amount of the PKC activator is provided in a dose from about 0.01-25 μg/m² IV.

Examples of suitable inhibitors of an arginine methyl transferase include one or more of the following: S-(5′-Adenosyl)-L-homocysteine; 7,7′-(carbonylbis(azanediyl))bis(4-oxidonaphthalene-2-sulfonate), sodium salt (AMI-1); 5′-Deoxy-5′(methylthio)adenosine;

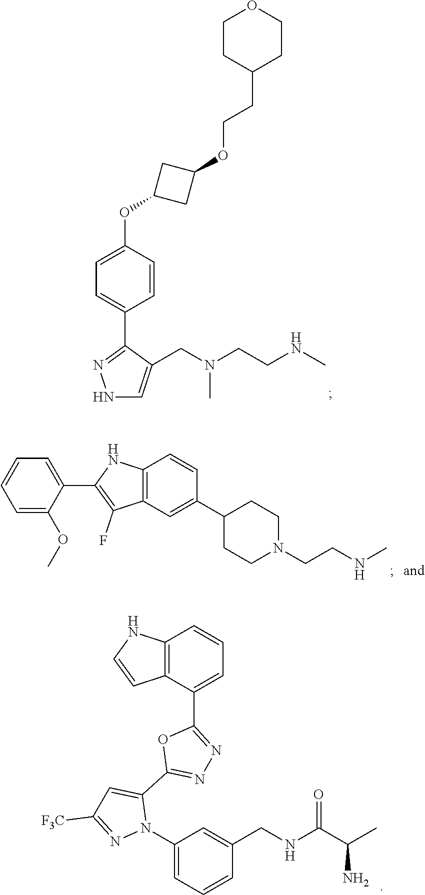 [SMILES: CNCCN(C)Cc1c[nH]nc1-c1ccc(O[C@H]2C[C@H](OCCC3CCOCC3)C2)cc1 ; CNCCN1CCC(c2ccc3[nH]c(-c4ccccc4OC)c(F)c3c2)CC1 ; C[C@@H](N)C(=O)NCc1cccc(-n2nc(C(F)(F)F)cc2-c2nnc(-c3cccc4[nH]ccc34)o2)c1]

Any of the aspects and embodiments described herein can be combined with any other aspect or embodiment as disclosed here in the Summary of the Invention, in the Drawings, and/or in the Detailed Description of the Invention, including the below specific, non-limiting, examples/embodiments of the present invention.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to or equivalent to those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and not intended to be limiting.

Other features and advantages of the application will become apparent from the following detailed description in conjunction with the examples.

### BRIEF DESCRIPTION OF THE DRAWINGS

FIGS. 1A-C demonstrate that CARM1 is a post-synaptic protein. In FIG. 1A, total 20 or 40 μg PSD fraction isolated from rat hippocampi were separated in SDS-PAGE and gels were stained with Coomassie solution. Arrow indicates CARM1 at 68 kDa. In FIG. 1B, immunoblot analysis to detect CARM1 protein levels in protein complexes extracted from PAGE gels using (A) anti-CARM1 (lane 1) or anti-serum pre-absorbed with CARM1 antigen (lane 2). In FIG. 1C, immunoblot analysis to detect CARM1 protein levels in whole tissue homogenates (WTH), synaptosomes (Syn), or One-Triton PSD fraction (10 μg/each fraction) (Mean±SEM, 3 independent experiments, **P<0.01, ***P<0.001, compared with WTH).

FIGS. 2A-D demonstrate that CARM1 co-localizes with PSD-95 in hippocampal neurons. In FIGS. 2A and B, cultured hippocampal neurons (14 DIV) were double-stained with anti-CARM1 and PSD-95 (a post-synaptic marker protein) or synapsin (a pre-synaptic marker), and then co-localization from overlay images was analyzed. Scale bar, 10 μm. In FIG. 2C, rat brain sections were labeled with antibodies against CARM1 or PSD-95 and gold particle labelings in the CA1 stratum radiatum of the hippocampus were observed using electron microscopy to show postembedded localization of CARM1 or PSD-95. Scale bar, 500 nm. In FIG. 2D, each CARM1 or PSD-95 labeling was counted to show pre- or post-synaptic positons of gold particles and the axodendritic distribution was quantitatively expressed.

FIGS. 3A-G demonstrate that reduction of CARM1 expression increases the complexity of dendritic arborization of cultured hippocampal neurons. In FIG. 3A, cultured hippocampal neurons were untreated or transduced with control scrambled (Cont), CARM1-, or PRMT1-specific siRNA at 9 DIV for 5 days. At 14 DIV, neurons were lysed and used for total RNA isolation and RT-qPCR. Relative CARM1 mRNA level was analyzed after normalization with GAPDH mRNA level. In FIG. 3B, immunoblot results with anti-CARM1, anti-PRMT1, and anti-Actin antibodies from hippocampal neurons treated as in FIG. 3A. In FIG. 3C, cultured hippocampal neurons treated as in FIG. 3A were fixed at 14 DIV and stained with MAP2, a dendritic marker. Whole cell shape is shown. Scale bar, 30 μm. In FIGS. 3D and 3E, quantitative analysis of TDBL (μm) and TDBTN from tracing images of neurons treated as in FIG. 3C. In FIGS. 3F and 3G, Sholl analysis of dendritic complexity in neurons treated as in (C) (Mean±SEM, N=40 neurons/condition from 3 independent experiments, *P<0.05, **P<0.01, ***P<0.001, compared with untreated condition).

FIGS. 4A-E demonstrate that treatment of CARM1 inhibitor promotes dendritic spine maturation in cultured hippocampal neurons. In FIG. 4A, cultured hippocampal neurons were transfected with GFP-Actin at 7 DIV and untreated or treated at 10 DIV with control media (untreated) or AMI-1 (a specific CARM1 inhibitor, 2.5 or 10 μM) for 4 days. At 14 DIV, neurons were fixed and dendritic spine morphology was visualized by confocal microscopy. Scale bar, 10 μm. Spine length (μm, FIG. 4B), spine head width (μm, FIG. 4C), or spine density (number of spines/10 μm dendrite length, FIG. 4D) was calculated and statistically analyzed. In FIG. 4E, dendritic spine shape was counted and expressed as % (Mean±SEM, N>100 spines from 3 independent experiments, **P<0.01, compared with untreated condition).

FIGS. 5A-E demonstrate that reduction of CARM1 expression modifies dendritic spine maturation in cultured hippocampal neurons. In FIG. 5A, cultured hippocampal neurons were untreated or transfected with GFP-Actin at 7 DIV. After 2 days, cells were transduced with control siRNA (Cont) or CARM1-specific siRNA and incubated for another 5 days. At 14 DIV, neurons were fixed and dendritic spine morphology was visualized by confocal microscopy. Scale bar, 10 μm. Spine length (μm, FIG. 5B), spine head width (μm, FIG. 5C), or spine density (number of spines/10 μm dendrite length, FIG. 5D) was calculated and statistically analyzed. In FIG. 5E, dendritic spine shape was counted and expressed as % (Mean±SEM, N>100 spines from 3 independent experiments, *P<0.05, compared with untreated condition).

FIGS. 6A-C demonstrate that CARM1 inhibition increases post-synaptic targeting of NR2B and PSD-95. In FIG. 6A, cultured hippocampal neurons at 10 DIV were treated with control media (untreated) or AMI-1 (10 μM) and incubated for 4 days. At 14 DIV, neurons were fixed and used for double-staining with NR2B and PSD-95 antibodies. Scale bar, 10 μm. In FIGS. 6B And 6C, synaptic cluster size (μm2) and synaptic clustering (number per 10 μm) were calculated and statistically analyzed (Mean±SEM, N>100 spines from 3 independent experiments, *P<0.05, **P<0.01, compared with untreated condition).

FIGS. 7A-E demonstrate that knock-down of CARM1 protein promotes post-synaptic clustering of NR2B and PSD-95 proteins. In FIGS. 7A-7C, cultured hippocampal neurons were untreated or transduced with control (Cont) or CARM1-specific siRNA at 9 DIV and incubated for 5 days. At 14 DIV, neurons were fixed and used for double-staining with CARM1 and synapsin (FIG. 7A), NR2B (FIG. 7B), or PSD-95 (FIG. 7C) antibodies. Scale bar, 10 μm. In FIGS. 7D and 7E, synaptic cluster size (μm2) and synaptic clustering (number per 10 μm) were calculated and statistically analyzed (Mean±SEM, 3 independent experiments, *P<0.05, **P<0.01, compared with untreated).

### DETAILED DESCRIPTION OF THE INVENTION

### Synaptogenesis

Synaptogenesis refers to the process by which synapses are formed between neurons. Two types of synaptogenesis occur after damage in the adult central nervous system (CNS): regenerative synaptogenesis and reactive synaptogenesis. Regenerative synaptogenesis occurs when injured axons begin sprouting and may result in the axon extending long distances in order to reach its original target, whereas reactive synaptogenesis occurs within 4-5 days of injury when nearby undamaged axons innervate synaptic sites that were previously activated by the injured axons.

### Protein Kinase C Family

The protein kinase C (PKC) family of enzymes is responsible for a multitude of cellular processes through the enzymes' ability to regulate proteins via signal transduction cascades. The members of this kinase family are structurally and functionally similar and are categorized into conventional (α, β1, βII and γ), novel (δ, ε, η, and θ), and atypical isoforms (ζ and λ). These isoforms have been implicated in a variety of diseases and pathological conditions. (See Mellor and Parker (1998) Biochem. J. 332(2): 281-292; Azzi et al. (1992) Eur. J. Biochem. 208:547-557; Cloud-Heflin et al. (1996) Eur. J. Biochem. 239: 796-804; and Mochly-Rosen et al. Nat. Rev. Drug Discov. 11: 937-957.)

The PKC ε and PKC α isozymes are responsible for increasing the synthesis of synaptic growth factors including BDNF, IGF, and NGF, thereby increasing the levels of these growth factors. Further, the PKC ε and PKC α isozymes are anti-apoptotic, i.e., they prevent and/or reduce neuronal and synaptic death. In one embodiment, PKC ε contributes more than PKC α towards the increase in the synthesis of synaptic growth factors including BDNF, IGF, and NGF. In one embodiment, PKC ε is more efficacious at preventing and/or reducing neuronal and synaptic death than PKC α.

### PKC Activators

In general, the present disclosure provides methods for regenerative synaptogenesis using PKC activators and inhibitors of arginine methyltransferases. As used herein, “protein kinase C activator” or “PKC activator” refers to a substance that increases the rate of the reaction catalyzed by protein kinase C, upregulates the expression of PKC (e.g., upregulates the expression of PKCα, PKC βII, PKC γ and/or PKC ε), or otherwise facilitates the activation of PKC.

The PKC activator may be any of bryostatin 1-20, α bryolog, neristatin, a polyunsaturated fatty acid, or any combinations thereof.

Bryostatins may be used in the methods of the present disclosure. The bryostatins are a family of naturally occurring macrocyclic compounds originally isolated from marine bryozoa. Currently, there are about 20 known natural bryostatins which share three six-membered rings designated A, B and C, and which differ mainly in the nature of their substituents at C7 (OR^{A}) and C20 (R^{B}). For example, in bryostatin 1, R^{A} is —C(═O)CH₃ (acetyl) and R^{B} is —OC(═O)CH═CH—CH═CH—C₃H₇.

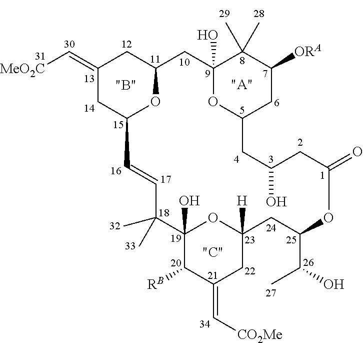

Bryostatin 1 and derivatives of bryostatin 1 are described in U.S. Pat. No. 4,560,774 (incorporated herein by reference). Examples of suitable bryostatins that may be used with the methods of the present disclosure include, bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17 bryostatin 18, bryostatin 19, and bryostatin 20.

Analogs of bryostatins, commonly referred to as bryologs, may also be used in the methods of the present disclosure. Bryologs are structural analogues of bryostatin. While bryostatin has two pyran rings and one 6-membered cyclic acetal, in most bryologs one of the pyrans of bryostatin is replaced with a second 6-membered acetal ring. This modification reduces the stability of bryologs, relative to bryostatin, for example, in both strong acid or base, but has little significance at physiological pH. Bryologs also have a lower molecular weight (ranging from about 600 to 755), as compared to bryostatin (988), a property which may facilitate transport across the blood-brain barrier. Examples of suitable bryologs include, but are not limited to, analogs and derivatives of bryostatins such as those disclosed in U.S. Pat. Nos. 6,624,189, 7,256,286 and 8,497,385 (the disclosures of which are incorporated herein by reference).

Polyunsaturated fatty acid esters (PUFAs or polyenoic fatty acids)) may be used in the methods of the present disclosure. A PUFA is a fatty acid containing more than one double bond. There are three classes of PUFAs, omega-3 PUFAs, omega-6 PUFAs, and omega-9 PUFAS. In omega-3 PUFAs, the first double bond is found 3 carbons away from the last carbon in the chain (the omega carbon). In omega-6 PUFAs the first double bond is found 6 carbons away from the chain and in omega-9 PUFAs the first double bond is 9 carbons from the omega carbon. As used herein, the term PUFA includes both naturally-occurring and synthetic fatty acids. A major source for PUFAs is from marine fish and vegetable oils derived from oil seed crops. Examples of PUFA's suitable for use in the methods of the present disclosure include, but are not limited to, esters of 8-[2-(2-pentylcyclopropylmethyl) cyclopropyl]-octanoic acid (DCPLA), as well as those described in U.S. Pat. No. 8,163,800 and in PCT publication WO 2010014585 A1.

Other examples of suitable PKC activators include potassium channel activators such as, for example, diazoxide.

Neristatins, such as neristatin 1, may be used in the methods of the present disclosure for treating lipid storage disorders.

Other suitable PKC activators include, but are not limited to, phorbol-12-myristate-13-acetate (PMA), okadaic acid, 1α,25-dihydroxyvitamin D3, 12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino)benzolactam(α-amyloid precursor protein modulator), cis-9-octadecenoic acid (oleic acid), ingenol 3-angelate, resiniferatoxin, L-α-Phosphatidyl-D-myo-inositol-4,5-bisphosphate, triammonium salt (PIP2), phorbol-12, 13-dibutyrate, 8(S-hydroxy-(5Z, 9E, 11Z, 14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12, 13-diacetate, phorbol-12, 13-didecanoate, 1,2-dipalmitoyl-sn-glycerol, 1-Stearoyl-2-linoleoyl-sn-glycerol, phorbol-12, 13-didecanoate, 1,2-dipalmitoyl-sn-glycerol, 1-stearoyl-2-linoleoyl-sn-glycerol, phorbol 12, 13-dihexanoate, prostratin and its analogs, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime)hexane (RHC-80267), (+/−)-1-oleoyl-2-acetylglycerol, 5(S),6(R), 15(S)-TriHETE (Lipoxin A4), (−)-Indolactam V, SC-9, SC-10, zoledronic acid monohydrate, 12-deoxyphorbo 13-angelate 20-acetate, 6-(N-decylamino)-4-hydroxymethylindole, 4aphorbol 12, 13-dibutyrate, 1,2-dihexanoyl-sn-glycerol, zoledronic acid disodium salt tetrahydrate, arachidonic acid methyl ester, arachidonic acid-d8.

### Arginine Methyltransferases

Protein arginine methyltransferases (PRMTs) transfer a methyl group from the cofactor S-adenosyl-L-methionine (SAM) to the terminal guanidino nitrogens of arginine on substrate proteins and can be divided into subtypes depending on the degree and position of methylation. (See Ferreira de Freitas et al., J. Med. Chem. 59:1176-1183 (2016)). Specifically, Type I and type II PRMTs convert arginine into monomethylarginine and further into asymmetric and symmetric dimethylarginine, respectively, while type III enzymes only monomethylate their substrates. (See id.).

Nine PRMTs (PRMT1, PRMT2, PRMT3, PRMT4, PRMT5, PRMT6, PRMT7, PRMT8, and PRMT9) have been identified in humans. In humans, PRMTs 1, 3, 4, 6, and 8 are type I enzymes, PRMT5 and PRMT9 are type II, and PRMT7 is the only known type III PRMT. (See id.).

PRMT6 methylates histone H3 at R2 and histones H4/H2A at R3 in vitro and is the major H3R2 methyltransferase in vivo. Moreover, PRMT6 is overexpressed in various types of human cancers such as breast, bladder, lung cancers (see Phalke et al., Nucleic Acids Res 40:9543-42 (2012); Yoshimatsu et al., Int. J. Cancer 128:562-73 (2011)) or melanoma (see Limm et al., Eur. J. Cancer 49:1305-13 (2013) and is directly implicated in the pathogenesis of neurodegenerative diseases (see Scaramuzzino et al., Neuron 85:88-100 (2015).

PRMT4 (also known as CARM1 (coactivator-associated arginine methyltransferase 1)) methylates Arg2, -17, and -26 of histone H3 and many non-histone proteins, including transcriptional coactivators and splicing factors, suggesting functions in transcriptional regulation and mRNA processing. (See Yang et al., Nat. Rev Cancer 13:37-50 (2013); Cheng et al., Mol. Cell 25:71-83 (2007); Lee et al., Proc. Natl Acad. Sci. USA 102:3611-3616 (2005)). PRMT4 has also been It has been implicated in leukemia and solid tumors. (See Vu et al., Cell Rep. 5:1625-38 (2013); Ou et al., Mol. Cancer Res. 9:660-70 (2011); and Frietze et al., Cancer Res. 68:301-306 (2008)).

### Inhibitors of Arginine Methyltransferases

Any suitable inhibitors of arginine methyltransferases can be used in any of the methods and compositions disclosed herein. By way of non-limiting example, these may include S-(5′ -Adenosyl)-L-homocysteine; 7,7′-(carbonylbis(azanediyl))bis(4-oxidonaphthalene-2-sulfonate), sodium salt (AMI-1); 5′-Deoxy-5′(methylthio)adenosine;

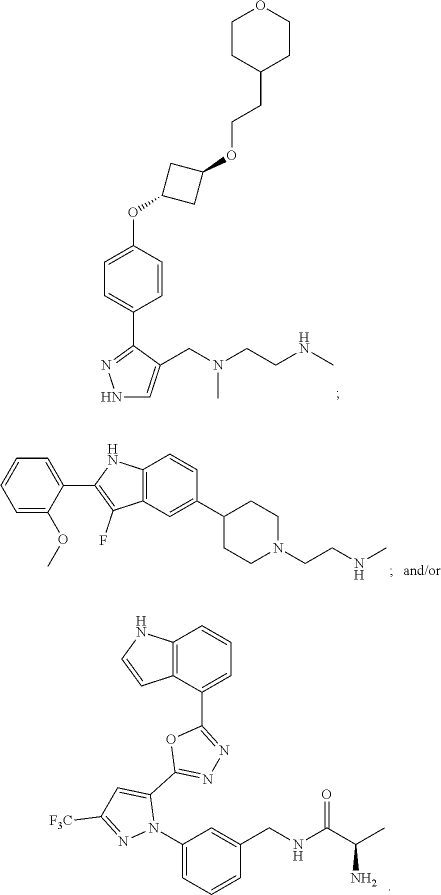 [SMILES: CNCCN(C)Cc1c[nH]nc1-c1ccc(O[C@H]2C[C@H](OCCC3CCOCC3)C2)cc1 ; CNCCN1CCC(c2ccc3[nH]c(-c4ccccc4OC)c(F)c3c2)CC1 ; C[C@@H](N)C(=O)NCc1cccc(-n2nc(C(F)(F)F)cc2-c2nnc(-c3cccc4[nH]ccc34)o2)c1]

(See, e.g., Sack et al., Biochem. J. 436:331-39 (2011); Mitchell et al., ACS Med. Chem. Lett. 6:655-59 (2015); and Ferreira de Freitas et al., J. Med. Chem. 59:1176-1183 (2016)).

Additionally, any arginine methyltransferases identified using fragment-based approaches can also be employed. (See Ferreira de Freitas et al., J. Med. Chem. 59:1176-1183 (2016)).

### Methods of Use

As used herein, “a pharmaceutically effective amount” is an amount of a pharmaceutical compound or composition having a therapeutically relevant effect.

Pharmaceutically effective amount for bryostatins and bryologs may be from about 0.0000001 to about 500 mg per kg host body weight per day, which can be administered in single or multiple doses. In some embodiments, the dosage level may be: from about 0.0000001 mg/kg to about 250 mg/kg per day; from about 0.0000005 mg/kg to about 100 mg/kg per day; from at least about 0.0000001 mg/kg to about 250 mg/kg per day; from at least about 0.00000005 mg/kg to about 100 mg/kg per day; from at least about 0.000001 mg/kg to about 50 mg/kg per day; or from about 0.00001 mg/kg to about 5.0 mg/kg per dose. In other embodiments, the dosage may be about 0.00000001 mg/kg to about 0.00005 mg/kg; 0.00005 mg/kg to about 0.05 mg/kg; about 0.0005 mg/kg to about 5.0 mg/kg per day; about 0.0001 mg/kg to about 0.5 mg/kg per dose; or 0.001 to 0.25 mg/kg per dose.

In certain embodiments, the dosing is from about 1 μg/kg (3-25 μg/m²) to 120 μg/kg (360-3000 μg/m²). In other embodiments, the dosing is from about 0.04-0.3 μg/kg (1 μg/m²) to about 1-10 μg/kg (25 μg/m²). In other embodiments, the dosing is from about 0.01 μg/m² to about 25 μg/m². In other embodiments, the dosing is from about 0.0002-0.0004 μg/kg to about 0.05-1 μg/kg.

When the PKC activator is a PUFA administered at a dosage of about 0.001 to 100 mg/kg; 0.01 to about 50 mg/kg; about 0.1 to about 10 mg/kg.

When the PKC activator present in the compositions used in the methods of the present disclosure is a bryostatin or bryolog, and the bryostatin or bryolog is used in an amount from about 0.0001 to about 1000 milligrams. For example, the bryostatin or bryolog is used in an amount from at least about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or about 1000.0 milligrams.

Pharmaceutically effective amount of the arginine methyltransferase inhibitor may be from about 0.0000001 to about 500 mg per kg host body weight per day, which can be administered in single or multiple doses. In some embodiments, the dosage level may be: from about 0.0000001 mg/kg to about 250 mg/kg per day; from about 0.0000005 mg/kg to about 100 mg/kg per day; from at least about 0.0000001 mg/kg to about 250 mg/kg per day; from at least about 0.00000005 mg/kg to about 100 mg/kg per day; from at least about 0.000001 mg/kg to about 50 mg/kg per day; or from about 0.00001 mg/kg to about 5.0 mg/kg per dose. In other embodiments, the dosage may be about 0.00000001 mg/kg to about 0.00005 mg/kg; 0.00005 mg/kg to about 0.05 mg/kg; about 0.0005 mg/kg to about 5.0 mg/kg per day; about 0.0001 mg/kg to about 0.5 mg/kg per dose; or 0.001 to 0.25 mg/kg per dose.

Determination of the appropriate pharmaceutically effective amounts is within the routine level of skill in the art.

The compositions used in the methods of the present disclosure may be administered via any suitable route; for example, orally, intraperitoneally, subcutaneously, intranasally, buccally, trans-dermally intramuscularly, intrarectally, intravenously, and by oral inhalation.

The compositions used in the methods of the present disclosure may be administered on a regimen of 1 to 4 times per day, and in some embodiments, the compositions are administered twice a week, once a week, once every two weeks, once every three weeks, once every four weeks, once every six weeks, once every eight weeks or even less frequently depending on the needs of the patient.

The compositions used in the methods of the present disclosure may be administered as part of a course of treatment lasting for about 1 to about 30 days; about 1 to about 90 days; about 1 to about 120 days; about 1 to about 180 days; about 1 to 365 days; one year; two years; three years; or for the patient's lifetime.

It will be understood, however, that the specific dose level and frequency of dosage for any particular host may be varied and will depend upon a variety of factors including the activity of the specific compound employed, the metabolic stability and length of action of that compound, the age, body weight, general health, sex, diet, mode and time of administration, rate of excretion, drug combination, the nature of the disorder, the severity of the particular disorder, and the host undergoing therapy.

### Effects of CARM1 Inhibition at Dendritic Synapses

Understanding the cellular and molecular mechanisms underlying synaptic formation will provide insight into not only learning and memory processes, but also the basis for neurodevelopmental and neurodegenerative disorders (1-5). Synapses, neuronal structures that form intercellular junctions between axon terminals and small protrusions called dendritic spines, accumulate information in the form of life-long alterations in their molecular and structural composition (6). Dendritic spines receive synaptic input from pre-synaptic axon terminals and regulate post-synaptic transmission (7). Spine morphology is highly correlated with synaptic function (8). Interestingly, spine structures are highly plastic, undergoing continuous change that modifies synaptic transmission and strength (9).

Dendritic spines contain a prominent thickening at the cytoplasmic surface of the post-synaptic membrane, called the post-synaptic density (PSD) (10, 11). The PSD is a dynamic region that consists of a variety of scaffolding proteins, such as members the membrane-associated guanylate kinase family including PSD-95; membrane receptors such as N-methyl-D-aspartate (NMDA)-receptors, α-amino-3-hydroxy-5-methyl-4-isoxazolepropionic acid (AMPA) receptors, and neurotrophic receptors; intracellular signaling molecules such as protein kinase C (PKC), calcium/calmodulin-dependent protein kinase 2 (CaMKII), and small GTPase family members; and structural proteins such as actin and microtubule components (11-14). The spatio-temporal regulation of the various synaptic proteins at the dendritic spine holds the key to understanding the mechanisms by which synaptic structures are remodeled and synaptic function is regulated.

Post-translational modification of proteins within signaling cascades, such as phosphorylation and methylation of synaptic proteins, is a fundamental and essential mechanism for regulating axonal elongation and dendrite branching (8, 15). For example, protein phosphorylation by PKCε regulates synaptogenic gene expression and rescues synaptic plasticity that is impaired in neurodegenerative diseases like Alzheimer's disease (16, 17). Activated PKCε modulates morphological changes in dendritic spines and enhances long-term memory by activating structural changes in the synaptic cytoskeleton (18-20) or by up-regulating HuD-mediated post-transcriptional controls (21, 22).

Arginine methylation of synaptic proteins, catalyzed by protein arginine methyltransferases (PRMTs), is another common post-translational modification that occurs in neuronal cells (23-25). Dysregulation and aberrant expression of PRMTs are associated with various disease states (24, 26, 27). The regulation and substrate specificity of PRMTs have been rapidly elucidated in the past few years, but the biological functions of PRMTs in the brain and the effects of arginine methylation of synaptic proteins by PRMTs remains unclear. Coactivator-associated arginine methyltransferase 1 (CARM1), also called PRMT4, modulates protein methylation in neurons (28, 29). CARM1 methylates HuD protein and plays an important role in mRNA processing (22, 30). It also inhibits neuronal differentiation in vitro (28). CARM1 activity is modulated upon phosphorylation by PKCε (22, 31). Taken together, these findings strongly suggest that regulation of CARM1 activity through PKCε may influence learning-specific synaptic formation in the brain. Despite data suggesting that CARM1 functions in neurons, no studies have further characterized the cellular localization and function of CARM1 on dendritic maturation and synaptic formation.

The hypothesis was tested that CARM1 is expressed in the rat brain, particularly in the PSD fraction, where it regulates dendrite and synaptic morphology. The effects of genetic and pharmacologic inhibition of CARM1 on dendritic complexity, spine number and density, and arborization, and the accumulation of excitatory synaptic proteins at synaptic sites in primary cultures of differentiating rat hippocampal neurons were examined.

CARM1 is a Post-Synaptic Protein that Clusters at Synapses in Hippocampal Neurons

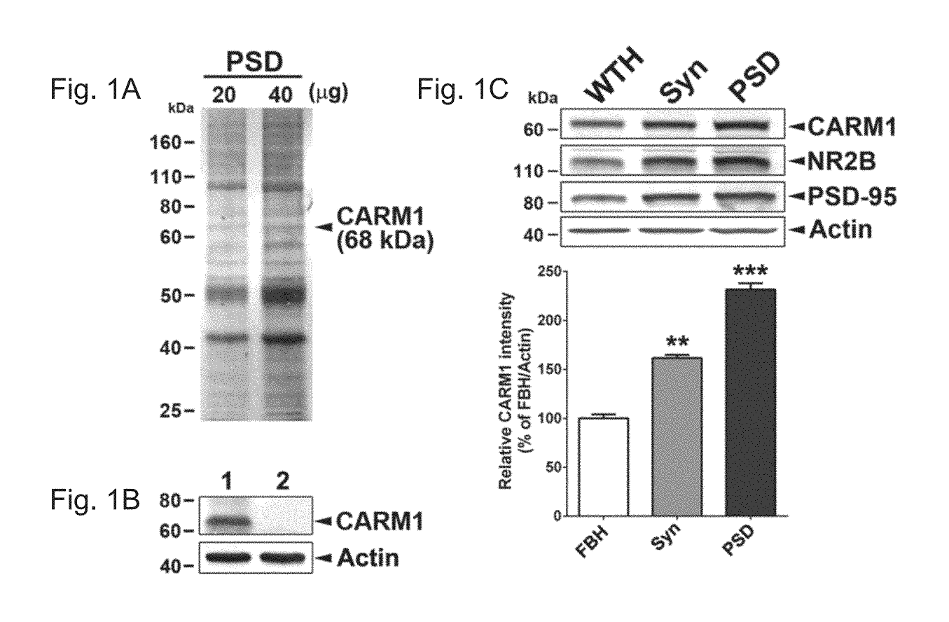

To understand CARM1 function at dendritic synapses in hippocampal neurons, CARM1 expression was first examined in brain tissue subcellular fractions. The One-Triton PSD fraction was separated by sodium dodecyl sulfate-polyacrylamide gel electrophoresis (SDS-PAGE), and a protein band of 68 kDa (FIG. 1A) was excised from the gel after Coomassie staining, extracted, separated by SDS-PAGE, and then immunoblotted using anti-CARM1 antibody (FIG. 1B), revealing strong CARM1 expression. To verify the specificity of the reaction, the anti-CARM1 antibody was pre-absorbed with CARM1 antigen. Pre-absorption of the CARM1 antibody completely abolished detection of CARM1 in the PSD fraction (FIG. 1B).

Next, whether CARM1 localizes to the PSD fraction in rat hippocampal neurons was tested. The relative enrichment of CARM1 in whole hippocampal tissue homogenate, synaptosomes, and PSD fractions treated once with Triton X-100 was tested by immunoblot analyses and compared with that of NR2B and PSD-95 proteins that are known to be specifically associated with the PSD fraction. Immunoblots revealed that the amount of CARM1 was enriched in the synaptosome and PSD fractions by 161.6% and 231.7%, respectively, compared to whole tissue homogenate (FIG. 1C). These results confirm that CARM1 is enriched in the PSD fraction.

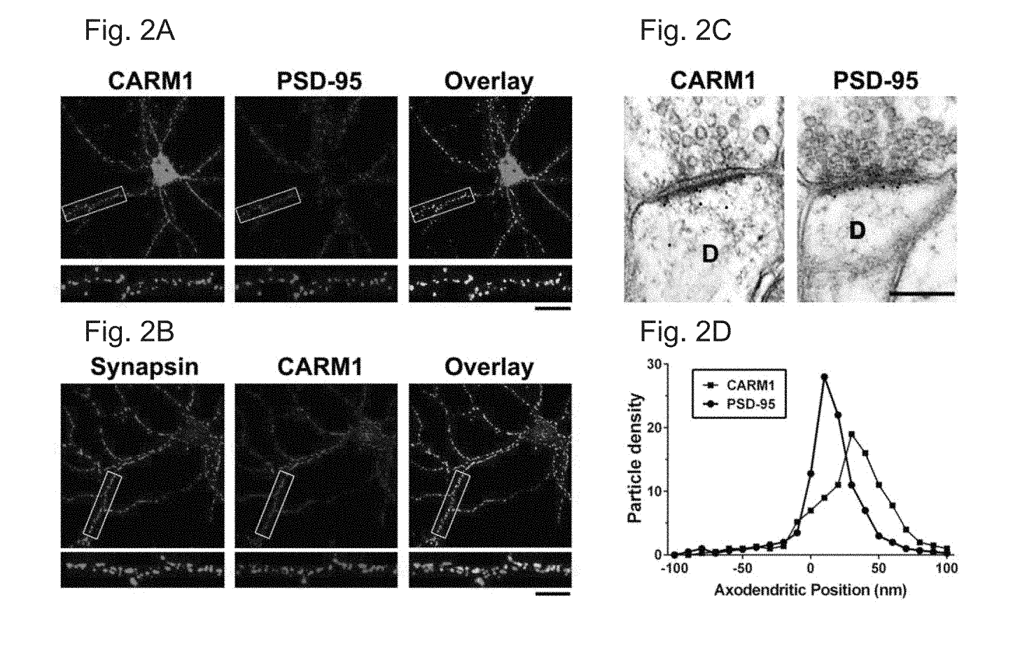

The subcellular distribution of CARM1 by fluorescence immunocytochemistry of dissociated rat hippocampal neurons was also examined. Dissociated hippocampal neurons were grown for 14 days in vitro, fixed, and double-stained with antibodies against CARM1 and the pre-synaptic marker synapsin or the excitatory post-synaptic marker PSD-95. Percentage of co-localization was calculated from overlay images (FIG. 2). High-resolution confocal microscope images showed that CARM1 formed clusters at sites that also contain PSD-95 (Mander's coefficient=0.726⊥0.041, FIG. 2A). However, CARM1 barely co-localized with synapsin (Mander's coefficient=0.292±0.034, FIG. 2B).

To verify that CARM1 is located at synapses in vivo and to determine whether CARM1 is predominantly located at the presynaptic or post-synaptic membrane, the distribution of CARM1 at synapses from the CA1 stratum radiatum of the hippocampus was examined with the use of immuno-electron microscopy. Brain sections were immunolabeled with an antibody against either CARM1 or PSD-95. The immunogold label in neurons for both antibodies was present primarily at excitatory synapses, identified by the presence of a Gray Type I PSD, round presynaptic vesicles, and a distinct synaptic cleft (FIG. 2C). Additionally, no labeled synapses were detected in control experiments in which sections were stained with antibodies that were preabsorbed with its antigen, respectively, or stained with secondary antibody alone. Thus the labeling of excitatory synapses accurately reflects localization of CARM1. Then, the axodendritic distribution of gold particles against CARM1 was quantitatively analyzed to determine whether the position of the label was over the presynaptic or postsynaptic compartment and was compared to that against PSD-95. The axodendritic distribution of the gold particles against CARM1 was analyzed by measuring the distance from the extracellular perimeter of the post-synaptic membrane to the center of the gold particle. The immunogold label against CARM1 was primarily over the postsynaptic membrane and compartment, confirming that over 87 percent of the gold particles were detected from the postsynaptic membrane and spine, with 15% of the label within 10 nm (the diameter of the gold particle) of the outer perimeter of the postsynaptic membrane and 81% within 50 nm (FIG. 2D). PSD-95 also preferentially labeled post-synaptic structures, with 89% of the gold particles over the postsynaptic compartment, with 41% of the label within 10 nm (the diameter of the gold particle) of the outer perimeter of the postsynaptic membrane and 83% within 50 nm (FIG. 2D). The prominent clustering of CARM1 at post-synaptic sites, revealed by higher co-localization with PSD-95, is consistent with its strong enrichment in the PSD fraction.

Knock-Down of CARM1 Expression Increases the Complexity of Dendritic Arborization of Cultured Hippocampal Neurons

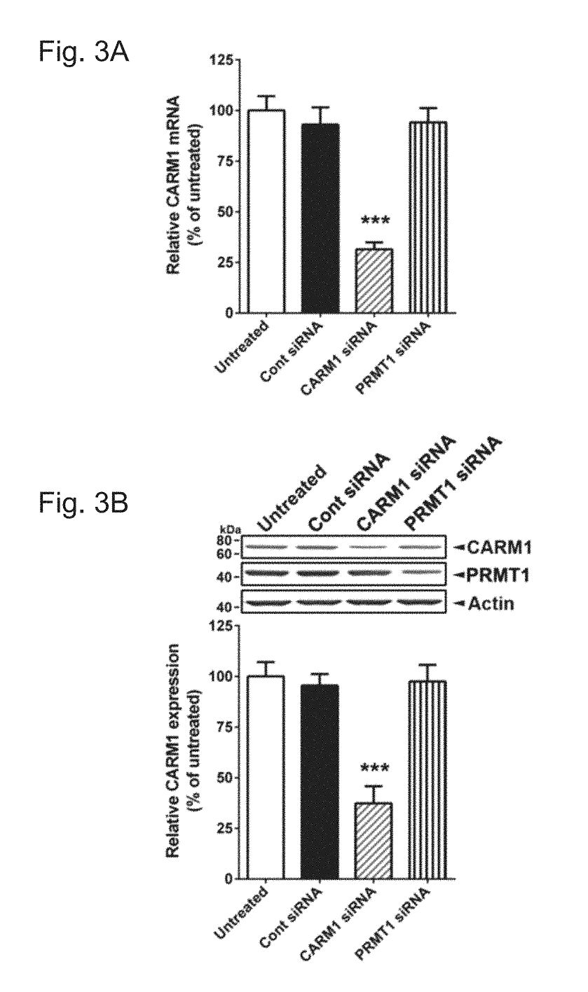

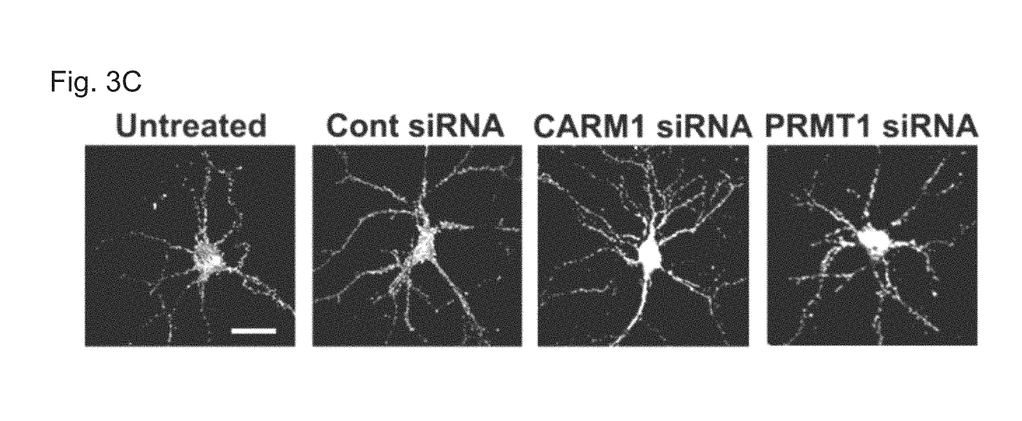

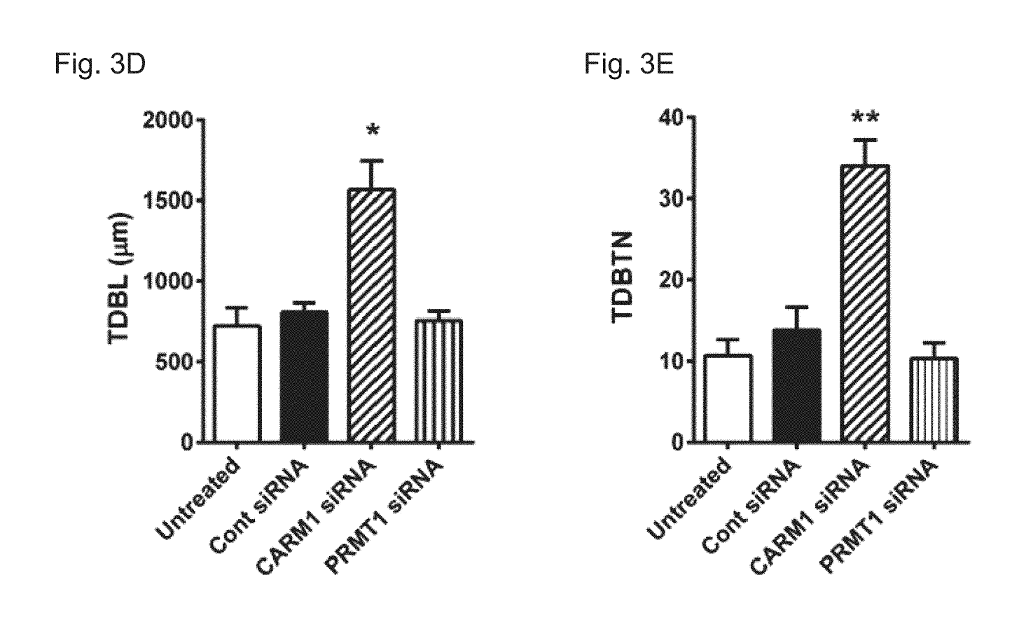

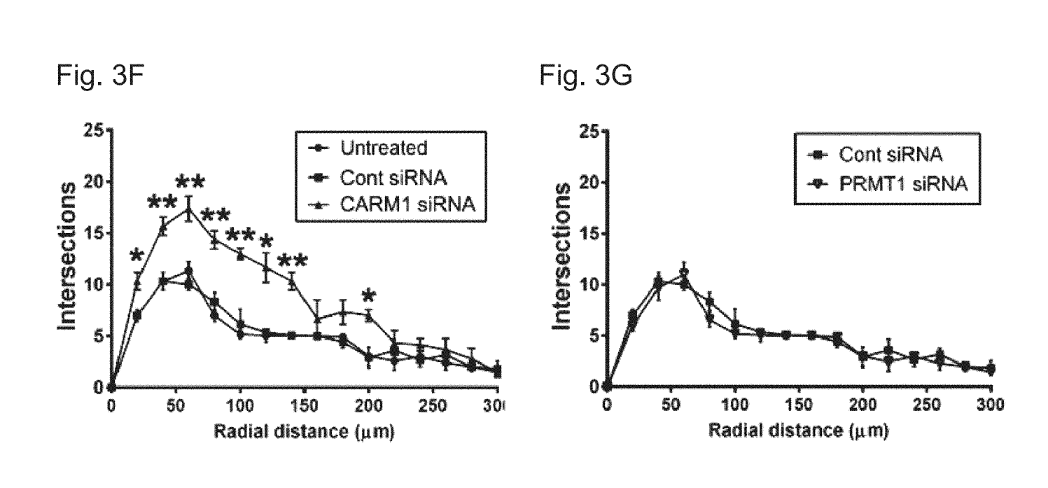

CARM1 inhibition by a specific CARM1 inhibitor or PKCε-mediated inhibition has been shown to promote the dendritic arborization of differentiating hippocampal neurons in vitro (22). To confirm whether CARM1 is important for dendritic maturation of dissociated hippocampal neurons, CARM1 expression was suppressed with specific siRNAs against CARM1 or PRMT1 (a predominant type I protein methyltransferase in mammalian cells, accounting for 85% of cellular PRMT activity, 32) as negative control. Using RT-qPCR, it was confirmed that transduction of CARM1-specific siRNA into neurons reduced CARM1 mRNA levels by 69% compared with untreated cells, and there was no difference in CARM1 mRNA levels in cells transduced with a scrambled control or PRMT1 siRNA compared with untreated cells (FIG. 3A). By immunoblot analysis, neurons transduced with CARM1-specific siRNA showed a 62% decrease in CARM1 protein levels, compared with neurons transduced with scrambled control siRNA or untreated cells. Neurons transduced with PRMT1-specific siRNA showed no difference in CARM1 protein levels compared with untreated cells, even that reduced PRMT1 protein levels by 55%, compared with untreated cells (FIG. 3B).

To determine the effects of CARM1 silencing on dendritic maturation of hippocampal neurons, neurons transduced with control, CARM1- or PRMT1-specific siRNA were fixed, stained with the dendritic marker MAP-2, and imaged by confocal microscopy. Neurons transduced with CARM1-specific siRNA showed increased dendritic branching and an enhanced dendritic arbor, but not with PRMT1-specific siRNA (FIG. 3C). To quantify the dendritic structure response to CARM1 silencing, images of the neurons stained with MAP-2 were traced in Neurolucida software for analysis of total dendritic branch length (TDBL) and total dendritic branch tip number (TDBTN). Transduction of neurons with CARM1-specific siRNA caused a dramatic increase in TDBL (1565.0±179.5 μm), compared with untreated (723.3±110.4 μm), control siRNAs (806.7±56.7 μm), or PRMT1 siRNAs (756.7±57.6 μm). TDBTN was also increased in neurons transduced with CARM1-specific siRNA (34.00±3.19), compared with untreated (10.67±1.99), control siRNA (13.83±2.75), or PRMT1 siRNAs (10.33±1.91) (FIGS. 3D and 3E). To quantify dendritic complexity, Sholl analysis was used to measure the number of dendritic branches that intersect in concentric circles at 20-μm intervals starting 20 μm from the center of the soma. The number of intersections was significantly increased by CARM1-specific siRNA, as far as 200 μm from the soma. The most dramatic effects were seen in the first 100 μm of the dendrites, where transduction of CARM1-specific siRNAs induced an approximately 1.7-fold increase in the number of intersections, compared with untreated or control siRNA (FIG. 3F). Transduction of PRMT1 siRNAs did not show any effect on dendritic complexity, compared with control siRNA (FIG. 3G). Together, these results demonstrate that CARM1 inhibition increases the total dendritic length and branching of cultured hippocampal neurons.

Treatment of Cultured Hippocampal Neurons with CARM1 Inhibitor Affects Spine Morphology

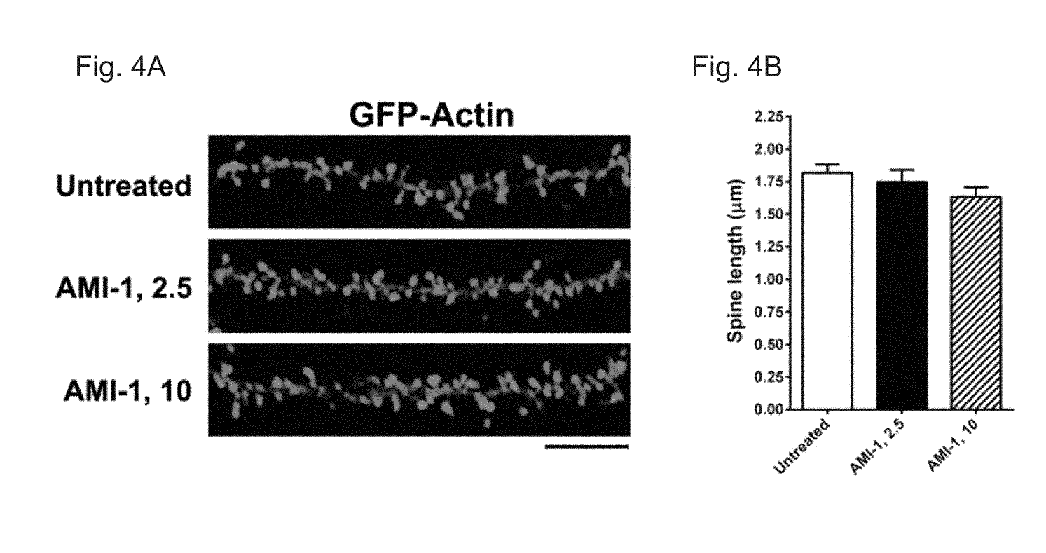

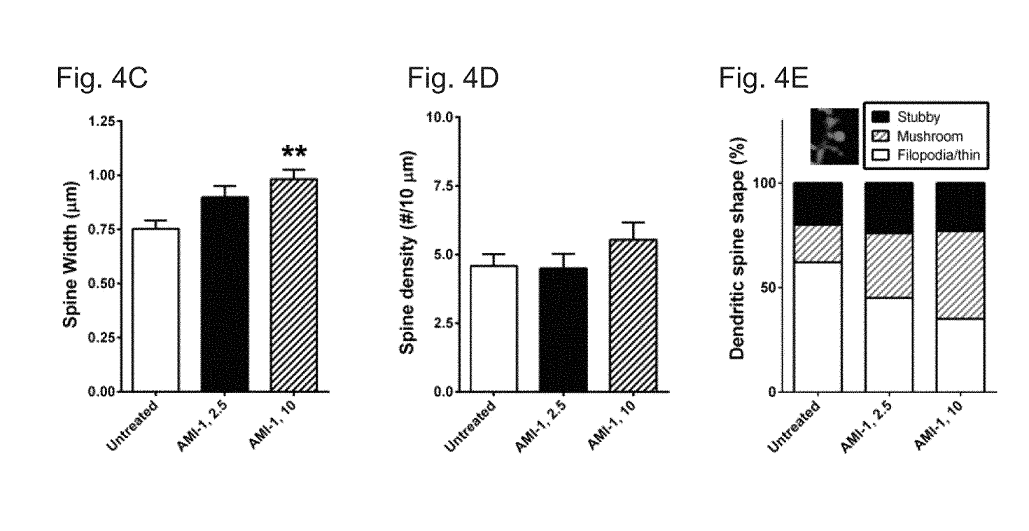

Since decreased expression of CARM1 led to an increase in dendritic complexity in cultured hippocampal neurons, whether inhibition of CARM1 signaling would have a similar effect on dendritic spine morphology was tested. Primary cultures of rat hippocampal neurons were transfected at 7 DIV with a GFP:actin plasmid to label the dendritic spines and then were untreated or treated with 2.5 μM or 10 μM of the CARM1-specific inhibitor AMI-1 for 4 days. Cultures were imaged by confocal microscopy (FIG. 4A) to evaluate spine length (from the base of the dendritic shaft to the tip of the spine) and width of each spine head (at its widest point). The length of the spines was slightly lower in cells treated with 10 μM AMI-1 (1.634±0.072 μm), but the difference was not statistically significant compared to untreated controls (1.819±0.064 μm) or cells treated with 2.5 μM (1.748±0.093 μm) (FIG. 4B). The width of dendritic spines was slightly higher in cells treated with 2.5 μM AMI-1 (0.899±0.052 μm) and significantly higher with 10 μM AMI-1 (0.982±0.044 μm, P=0.0085), compared with untreated control cells (0.753±0.038 μm) (FIG. 4C). There was no significant difference in spine density (the number of spines per 10 μm dendrite) along the dendritic shaft: untreated (4.58±0.43), 2.5 μM AMI-1 (4.49±0.54), and 10 μM AMI-1 (5.54±0.62) (FIG. 4D). Spine shapes were investigated using a morphometric analysis that takes into account the ratio of spine head and neck to the spine length to categorize the spines into 3 different types (stubby, mushroom, and filopodia, which includes long, thin-type spines). CARM1 inhibition with AMI-1 significantly increased the proportion of mushroom-type spines (P=0019), and led to a dramatic decrease the proportion of filopodia-type, including thin-type, spines (P=0.0027) (FIG. 4E).

Knock-Down of CARM1 Expression Alters Spine Morphology

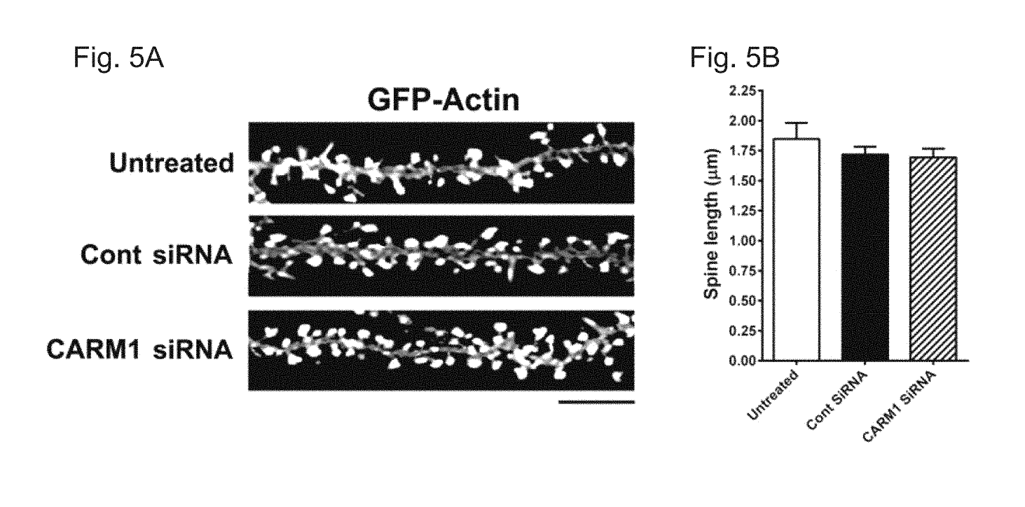

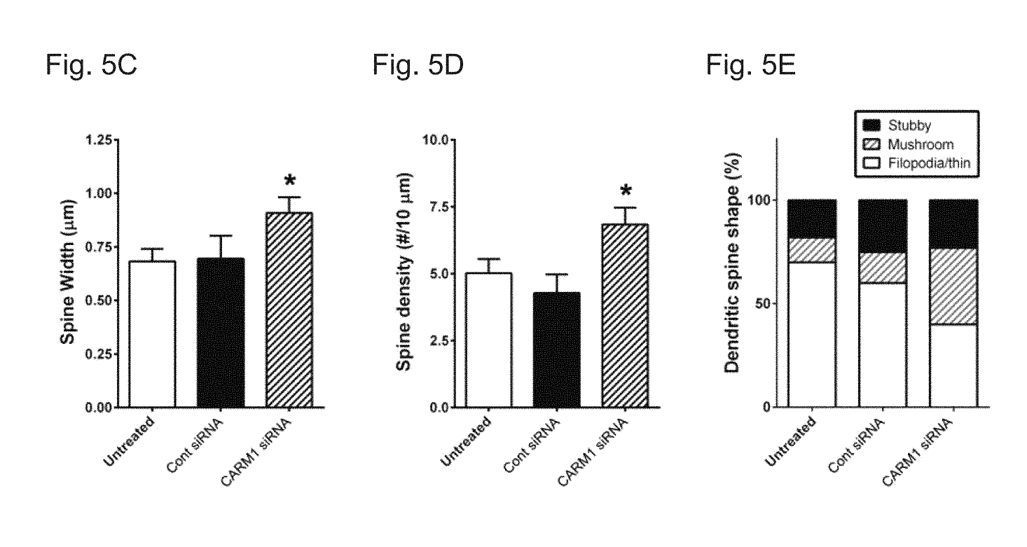

The effects of CARM1-specific siRNA on dendritic spine morphology were also examined. Primary hippocampal neurons were transfected at 7 DIV with a GFP:actin plasmid to label dendritic spines and then were untreated or transduced with control siRNA or CARM1-specific siRNA for 5 days and imaged by confocal microscopy (FIG. 5A). Spine length was not significantly different between the three groups: untreated (1.848±0.133 μm), control siRNA (1.719±0.064 μm), or CARM1-specific siRNA (1.694±0.0723 μm) (FIG. 5B). The width of the dendritic spines was significantly greater in cells transduced with CARM1-specific siRNA (0.909±0.072 μm, P=0.041), compared with untreated (0.683±0.059 μm) or control siRNA (0.708±0.107μm) (FIG. 5C). In addition, dendritic spine density was statistically significantly higher in the CARM1-specific siRNA group (6.84±0.62, P=0.0294) compared with the untreated (5.02±0.54) or control siRNA groups (4.28±0.69) (FIG. 5D). Transduction of neurons with CARM1-specific siRNA also affected dendritic spine shape, causing a dramatic increase in the proportion of mushroom-type spines (approximately 37%, P=0.0061), compared with untreated (12%) or control siRNA transduced (15%) cells. Conversely, the proportion of filopodia/thin-type spines was significantly lower in neurons transduced with CARM1-specific siRNA (approximately 40%, P=0.0196), compared with untreated (70%) or control siRNA transduced (60%) cells (FIG. 5E). These results are consistent with the changes in dendritic spine morphology seen with AMI-1 inhibition of CARM1, further supporting a role for CARM1 signaling in spine maturation during hippocampal neuronal differentiation.

CARM1 Inhibition Increases Post-Synaptic Targeting of NR2B and PSD-95

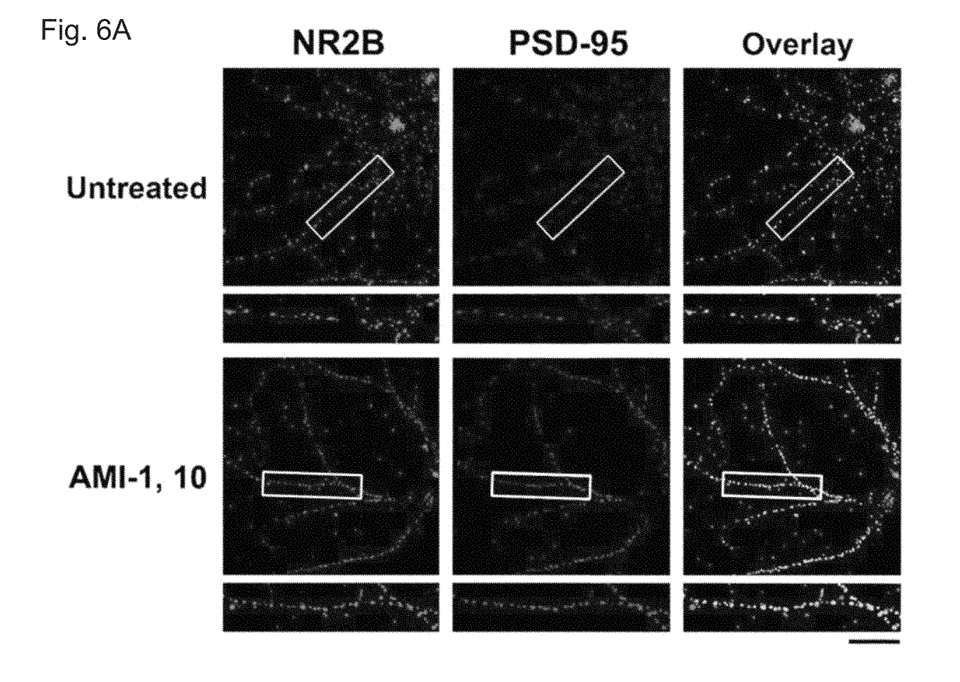

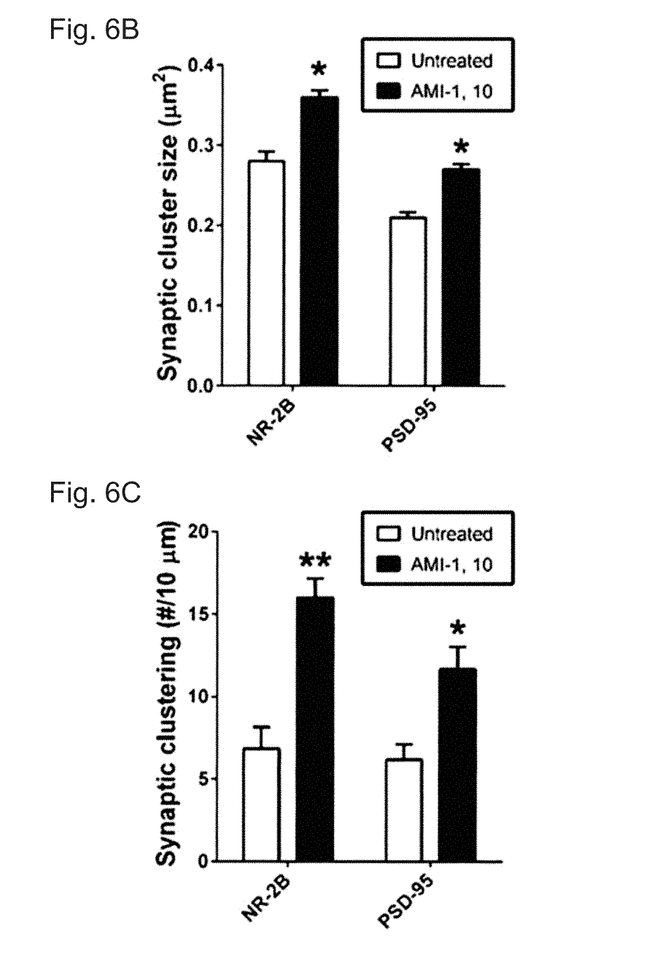

Spine morphological categories are highly correlated with synaptic function (33). CARM1 inhibition was found to promote elaboration of dendrites. Therefore, it was examined whether CARM1 inhibition using the pharmacological inhibitor AMI-1 or genetic knock-down with specific siRNA affects clustering of synaptic proteins, including synapsin (pre-synaptic) or NR2B and PSD-95 (post-synaptic), that mediate signal transmission and information processing at synaptic sites (34, 35). Primary hippocampal neurons at 10 DIV were untreated or treated with 10 μM AMI-1. After a 4-day incubation, neurons were fixed and double-stained for NR2B and PSD-95, and the average cluster sizes and the number of synaptic clusters for each protein were evaluated (FIG. 6A). Inhibition of CARM1 signaling by AMI-1 treatment led to an increase in the synaptic cluster sizes (FIG. 6B) and the number of clusters (FIG. 6C) of NR2B and PSD-95 compared to untreated cells. There was no difference between AMI-1 treated and untreated cells in synaptic cluster size or number for the pre-synaptic synapsin protein.

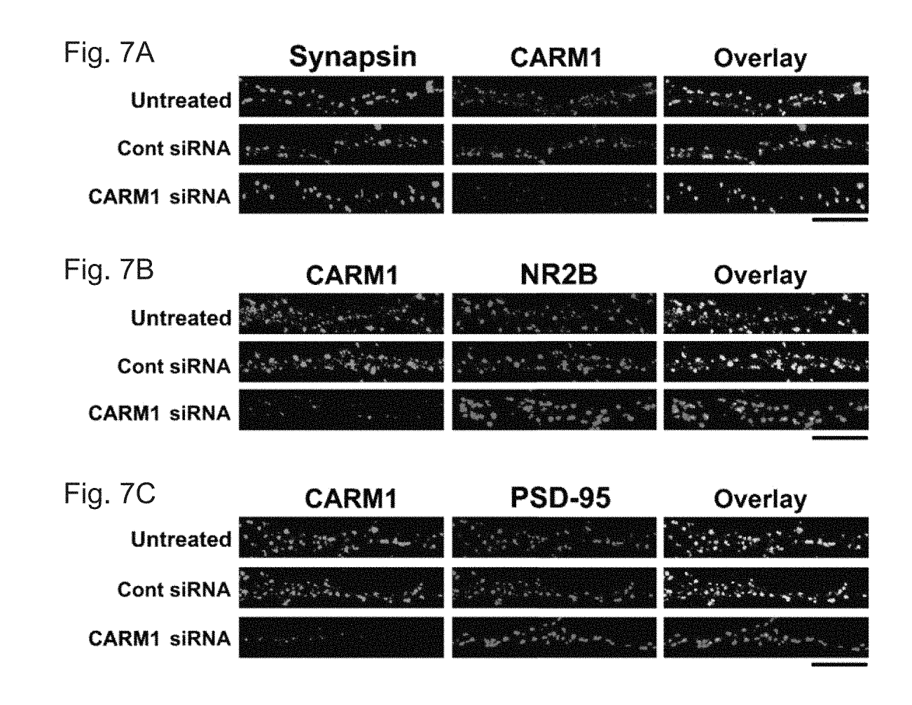

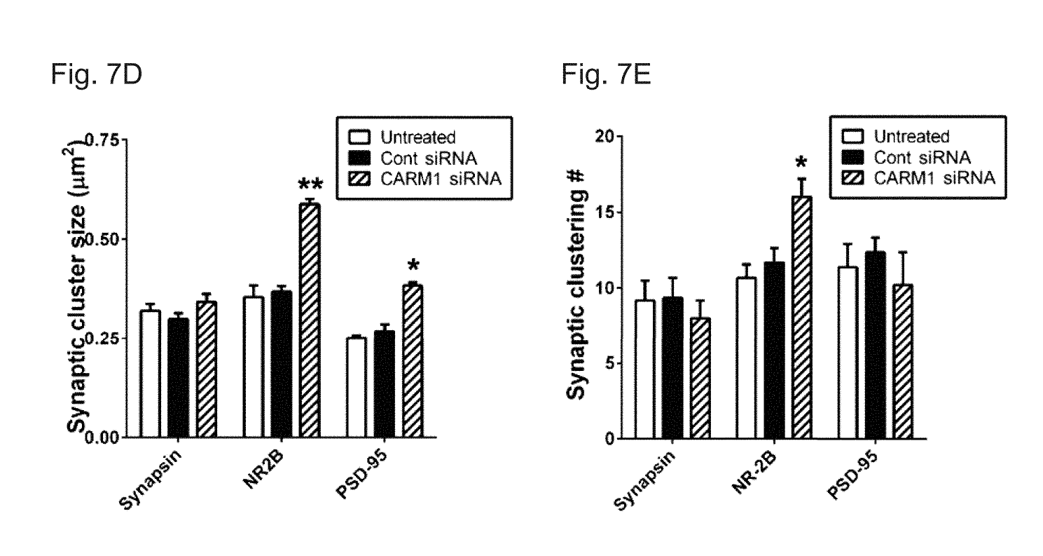

These findings were confirmed in neurons transduced with CARM1-specific siRNAs. Untreated hippocampal neurons or neurons transduced with control or CARM1-specific siRNA were double-stained for CARM1 and synapsin (FIG. 7A), CARM1 and NR2B (FIG. 7B), or CARM1 and PSD-95 (FIG. 7C). Immunocytochemical data clearly showed that transduction of CARM1-specific siRNA into hippocampal neurons decreased CARM1 expression (FIGS. 7A-7C). Consistent with the results seen after CARM1 inhibitor treatment, there was no difference in either the synaptic cluster size or cluster number of synapsin (FIGS. 7A, 7D, and 7E). By contrast, siRNA suppression of CARM1 expression led to a significant increase in NR2B cluster size and number compared with untreated or control siRNA-transduced cells (FIGS. 7B, 7D, and 7E). CARM1-specific siRNA transduction also led to a significant increase in the synaptic cluster size not in cluster number of PSD-95 compared with untreated or control siRNAs-transduced cells (FIGS. 7C, 7D, and 7E). Taken together, these data imply that regulation of CARM1 signaling is necessary for clustering of post-synaptic proteins at synapses of hippocampal neurons.

Discussion

CARM1 was found to be expressed in the PSD fraction of rat hippocampal neurons, confirmed by a series of SDS-PAGE and immunoblot analyses. It was also found that CARM1 is highly enriched in the PSD fraction and that it is present in clusters that co-localize with PSD-95, a post-synaptic marker, in dissociated hippocampal neurons and in dissected rat hippocampi, two main criteria for a protein to be considered a component of the PSD (10). Additionally, it has also demonstrated that inhibition of CARM1 activity, either pharmacologically or through siRNA knock-down, significantly increased dendritic complexity, with greater primary dendritic elongation and number of branches; induced dendritic maturation, as evidenced by an increase in mushroom-type spines; and enhanced clustering of the post-synaptic proteins NR2B and PSD-95 at synapses of differentiating hippocampal neurons in vitro.

CARM1 is a representative PRMT that inhibits neuronal differentiation in vitro (29). Despite data indicating that CARM1 functions in cellular systems, the role of CARM1 protein at the synapse in neurons has remained elusive. CARM1 may be involved in multiple steps during neuronal differentiation, and may be particularly important for the regulation of dendritic maturation and functional synapse formation. CARM1 has been shown previously to modulate mRNA stability and expression of synaptic genes through mRNA-binding proteins. It directly methylates HuD, an mRNA binding protein (30), and inhibits expression of neuronal genes including neurotrophic factors (NTF), such as brain-derived neurotrophic factor (BDNF), at the post-transcriptional level (22, 28, 29). NTF signaling, including that of mature BDNF, is important for dendritic spine formation and synaptogenesis (36-38). These findings suggest that CARM1 inhibition may be required for HuD-mRNA binding, stability, and expression of neuronal genes that are directly linked to dendritic synaptogenesis. Previous studies showed that treatment of a specific CARM1 inhibitor AMI-1 to hippocampal neurons in vitro increased HuD-mRNA binding and the stability and expression of NTFs, including BDNF, in hippocampal neurons (22). A growing body of evidence supports an important regulatory role for CARM1 in synapse function in the hippocampus, an area of the brain critical for learning, memory, and higher-level cognition.

CARM1 methylates diverse protein substrates and regulates intermolecular interactions and subcellular protein localization (39, 40), suggesting that it may not only affect gene expression but also protein function in the dendritic spines of hippocampal neurons, in turn affecting dendrite structure and dendritic spine morphology. Dendritic spines are structurally dynamic and undergo continuous remodeling even after complete maturation, reflecting modifications in synaptic strength and neural circuits (13, 41). During the fundamental processes of spine growth and shrinkage, polymerization and depolymerization of actin filament is required (42). A variety of small GTPases such as Rac1, Cdc42, and Ras are critical regulators of actin polymerization that influence spine number and morphology (43, 44). Cortactin directly binds to actin and phosphorylation-induced cortactin promotes the formation of dendritic protrusions (45). Therefore, future studies will investigate whether small GTPases and/or other actin-regulating factors lie downstream of CARM1 signaling, and how CARM1 may regulate these factors to modulate dendritic architecture and spine morphology and function.

In addition to regulating synaptic gene and protein expression, CARM1 may influence dendritic spine structure by regulating protein-protein interactions in the PSD. Interestingly, spine structures continuously change, reflecting the plasticity of synapses and the modification of synaptic transmission and strength. Changes in the molecular interactions among post-synaptic proteins in the PSD are required for morphological changes related to synaptic plasticity. PSD-95 is a MAGUK family member that interacts with NR2B. Upon neuronal activation, PSD-95 interacts with NR2B and helps stabilize its localization at post-synaptic membranes, thereby strengthening post-synaptic transmission (46). Inhibition of CARM1 signaling increases the cluster sizes of NR2B and PSD-95 at synapses in cultured hippocampal neurons, suggesting that CARM1 inhibits synaptic clustering of PSD proteins and alters signal transmission in hippocampal neurons. This loss of PSD protein clustering has been strongly correlated with disrupted learning behavior in mice (47). CARM1 can methylate neuronal proteins that contain an RXR motif and inhibit specific protein-protein interactions (24, 48). Proteomic analyses to identify post-synaptic proteins containing the RXR motif as potential CARM1 substrates in the PSD may help reveal how CARM1 modulates dendritic morphogenesis and whether CARM1-induced methylation of post-synaptic proteins interferes with synapse maturation.

Post-translational modification of synaptic proteins is an important process underlying long-lasting memory (49). CARM1 activity is specifically modulated by protein phosphorylation (22, 31). PKC can directly phosphorylate CARM1 and abrogate CARM1 activity (30, 31), including decreasing HuD methylation (22). During neuronal differentiation or neural activation, activated PKCε enhances post-transcriptional activation of synaptic genes and promotes dendritic maturation, while CARM1 inhibits further activation or dendritic maturation. The spatio-temporal regulation of synaptic proteins by CARM1 activation or inhibition at the PSD level may be the key to elucidating the mechanism through which synaptic structures are remodeled. Taken together, these findings strongly suggest that inhibition of CARM1 activity, either directly with a specific inhibitor or indirectly via PKCε-mediated modification, may influence learning-specific synaptic formation at the post-translational level in the hippocampus. Further studies using specific CARM1 inhibitors are required to determine how CARM1 activity is regulated in vivo.

In conclusion, CARM1 is enriched in the PSD and co-localizes with post-synaptic proteins in differentiating hippocampal neurons. The findings suggest that inhibition of CARM1 activity and down-regulation of CARM1 expression potentiates dendritic complexity and spine maturation during neuronal differentiation, which induces synapse formation and would affect the synaptic plasticity essential for learning and memory. Therefore, abnormal activation of CARM1 could contribute to neurological disorders and inhibition of CARM1 may be a potential therapeutic approach to recover synaptic dysfunction in age-related or neurodegenerative diseases.

The invention having been described, the following examples are offered by way of illustration and not limitation.

### EXAMPLES

### Example 1

### Experimental Procedures

Animals were carefully handled following the guideline and all animal experiments were approved by the West Virginia University Animal Care and Use Committee.

### Isolation of Hippocampal Subcellular Protein Fractions

Synaptic subcellular fractions were isolated from rat hippocampal tissue homogenates as described previously (50) with minor modifications. Six rat hippocampi were combined and homogenized in solution containing 0.32 M sucrose, 1 mM NaHCO3, 1 mM MgCl2, 0.5 mM CaCl2, 0.1 mM phenylmethylsulfonyl fluoride, and 1 mg/L leupeptin. This suspension was placed on a discontinuous sucrose gradient composed of 0.85 M, 1.0 M, and 1.2 M sucrose fractions, and separated by centrifugation for 2 hr at 82,500×g in a TLA-120.1 rotor in a Beckman Coulter Optima Max-XP ultracentrifuge. The synaptosome fraction at the 1.2 M and 1.0 M sucrose interface was collected and extracted with 0.5% Triton X-100 for 15 min. This fraction was pelleted by centrifugation at 36,800×g for 45 min in a TLA-120.1 rotor to yield the “One-Triton” PSD fraction and subjected to SDS-PAGE analyses, followed by Coomassie staining (Bio-Rad). Protein concentrations of each fraction were determined by the bicinchoninic acid (BCA, Thermo Scientific) method and subjected to immunoblot analysis, as described below.

### Hippocampal Neuron Culture and Treatment

Cultures of dissociated hippocampal neurons from embryonic day 18 rats were prepared as previously described (51). Cultured neurons at 10 days in vitro (DIV) were exposed to arginine N-methyltransferase inhibitor-1 (AMI-1; 2.5 or 10 μM, Enzo Life Sciences) in plating media minus additional glutamate for the indicated times.

### Immunoblot Analysis

Cultured neurons were lysed in cold lysis buffer (10 mM Tris-HCl, pH 7.4, 5 mM EDTA, 1% Triton x-100, 10% glycerol, 1 mM CaCl2, 1 mM MgCl2, 1× complete protease inhibitor cocktail, and 1× phosphatase inhibitor cocktail (Thermo Scientific)) for 1 hr at 4° C. After protein quantitation using the BCA method, equal quantities of cell lysates, tissue homogenates, or brain subcellular fractions were run on 8% or 10% SDS-PAGE gels and transferred to nitrocellulose membranes. Membranes were blocked for 1 hr in 5% nonfat milk in TBST (10 mM Tris, pH 7.5, 200 mM NaCl, 0.2% Tween-20) and incubated with rabbit anti-CARM1 (1:1,000, Cell Signaling), rabbit anti-PRMT1 (1:500, Fisher Scientific), rabbit anti-NR2B (1:2,000, Millipore), or rabbit anti-PSD-95 (1:1,000, Cell Signaling) antibodies. The membranes were washed in TBST and incubated with horseradish peroxidase (HRP)-conjugated goat anti-rabbit secondary IgG (1:10,000, Jackson Immunoresearch Laboratories). Bound antibodies were detected by Enhanced Chemiluminescence Substrate and exposed to X-ray film (Thermo Scientific). As a control, membranes were stripped and re-probed with primary antibody against mouse anti-Actin (1:5,000, Sigma-Aldrich) and HRP-conjugated goat anti-mouse IgG (1:10,000, Jackson Immunoresearch Laboratories). Levels of immunoreactivity for each band were assessed by densitometric analysis of films using an HP Scanjet densitometer and Image-J image analysis system software (1.44a, NIH), and then normalized to Actin levels.

### Electron Microscopy and Quantification

Three-month-old rats were anesthetized with a mixture of ketamine HCl (100 mg/kg body weight) and xylazine (20 mg/kg body weight). The rats were transcardially perfused with saline followed by perfusion with 4% paraformaldehyde and 0.5% glutaraldehyde in 0.1 M phosphate buffer (PB), pH 7.4. After perfusion, the brains were removed and post-fixed in the same fixative overnight at 4° C. The fixed brains were washed 4 times in PB and once in PB containing 4% glucose, then stored overnight in PB containing 4% glucose. The brain was cut into ˜350 μm sections on a vibratome and the sections immersed in PB containing 4% sucrose. The sections were then washed in 10% and 20% glycerol in PB for 1 hr each, then in 30% glycerol in PB overnight. A portion of the CA1 region of the hippocampus was cut from the sections and embedded by freeze substitution. The tissue was plunge frozen in an AFS freeze substitution instrument (Leica, Vienna, Austria) by liquid nitrogen immersed in 1.5% uranyl acetate in methanol for 27 hr at −90° C., and then warmed to −45° C., rinsed in anhydrous ethanol.

Sections were cut into ˜100 nm thickness and mounted on mesh grids. The sections were etched in 0.1% sodium borohydride, 50 mM glycine with Tris-buffered saline including Triton X-100 (TBS-T, 50 mM Tris-Cl, pH 7.4, 150 mM NaCl, 0.1% Triton X-100) for 10 min. The sections were washed in TBS-T twice for 5 min each and blocked with TBS-T including 2% bovine serum albumin for 30 min. The sections were incubated overnight with mouse anti-CARM1 (1:200, Cell Signaling) or mouse anti-PSD-95 (1:100, Cell Signaling). Control sections were incubated overnight with either antibodies that had been preabsorbed with the specific blocking peptides or with the secondary antibodies. After rinsing in blocking buffer 3 times, incubation in primary antibody, the sections were then incubated with 10 nm gold-conjugated secondary antibodies (1:20 dilution in blocking buffer with 0.5% polyethylene glycol, Jackson Immunoresearch Laboratories) for 2 hr. After rinsing in distilled water and counterstaining in 2% uranyl acetate and in Reynolds lead citrate, the sections were observed on a Phillips 300 transmission electron microscope. Excitatory synapses were identified by the presence of a distinct synaptic cleft, round presynaptic vesicles, and a Gray Type 1 PSD. All labeled synapses were photographed.

Quantification of the distribution of gold label was conducted on images of immunolabeled tissue derived from two rats. The digitized images were imported into NIH ImageJ software for further analysis by an investigator blind to the experimental conditions. To determine the axodendritic distribution of gold particles, the distance between the outer perimeter of the postsynaptic membrane and the center of each gold particle was measured. All gold particles within 100 nm of the postsynaptic membrane towards either the presynaptic or postsynaptic side were measured. A total of 13 synapses and 54 gold particles were analyzed for synapses labeled with CARM1, and 17 synapses and 78 gold particles were analyzed for synapses labeled with the PSD-95. Distances to gold particles on the presynaptic side were given negative values, and distances to gold particles on the postsynaptic side were given positive values. To analyze the tangential distribution of labeling along the PSD, the distance between the gold particle and the midline of the PSD was measured. This distance was normalized as a percentage of the total distance between the midline and the lateral edge of the PSD.

### Transfection and CARM1 Gene Silencing

To identify the effects of genetic knock-down of CARM1 on dendritic morphology and synaptic clustering of synapsin, NR2B, and PSD-95, hippocampal neurons were first transfected with the construct pCAG-mGFP-Actin [a gift from Ryohei Yasuda (Addgene plasmid #21948)] at 7 DIV with Lipofectamine 2000 (Invitrogen) following the manufacturer's protocol. After a 2-day incubation, neurons were untreated or transduced with rat CARM1- or PRMT1-specific siRNA or scrambled control siRNA lentiviral particles (5×103 viral particles/μl, Santa Cruz) at a multiplicity of infection (MOI) of 4 to ensure efficient infection following the manufacturer's protocol, followed by overnight incubation. After a media change with plating media minus glutamate, cells were incubated for an additional 4 days, and then a portion of the cells was either lysed and used for total RNA isolation and RT-qPCR experiments, lysed and used for immunoblot analysis, or fixed for immunocytochemical analysis.

### Real-Time Quantitative RT-PCR

Total RNA was isolated using the RNeasy mini kit (Qiagen) per the manufacturer's protocol. For the RT reaction, 500 ng of total RNA was reverse transcribed using oligo(dT) primer and Superscript III (Invitrogen) at 50° C. for 1 hr. Real-time qPCR was performed for 40 cycles with SYBR Green 1 PCR master mixture and processed on LightCycler 480 II (Roche) machine using specific primer sets against rat CARM1, PRMT1, or GAPDH (all from Qiagen). Reactions were run in triplicate for each sample and a dissociation curve was generated. Threshold cycles (Ct) for CARM1 amplification were normalized to the housekeeping gene GAPDH (ΔCt) and every experimental sample was referred to its control (ΔΔCt). Relative expression change values were expressed as 2-ΔΔCt.

### Immunocytochemistry and Quantification of Dendritic Branching

Cultured neurons were treated as described above, fixed, and then incubated with primary antibodies against mouse anti-MAP-2 (1:1,000, Abcam) and Alexa 488-conjugated anti-mouse secondary IgG (1:1,000, Invitrogen) as previously described (22). The immunostained neurons were viewed on a Zeiss LSM 710 laser-scanning confocal microscope. Neurons from blindly predetermined locations on the coverslip were imaged in Z-sections, and maximal projections of the images were used for analysis. To quantify changes in dendritic structure, images of neurons stained with MAP-2 were opened in Neurolucida software (MicroBrightField Bioscience) and the dendrites were traced and analyzed by a person blinded to the treatment condition. Tracings were then automatically analyzed in Neuroexplorer software (MicroBrightField Bioscience) for TDBL and TDBTN. Neuroexplorer was also used for Sholl analysis to quantify the number of dendrite intersections in concentric rings centered about the soma beginning at a radius of 20 μm from the center of the soma and at 20-μm intervals thereafter. Forty neurons derived from 3 independent cultures were randomly selected, traced, and analyzed for each condition.

### Immunocytochemistry and Image Acquirement

Cultures neurons were treated as described above and then fixed in 2% paraformaldehyde/4% sucrose/PBS for 10 min at room temperature (RT) followed by methanol for 20 min at −20° C. The fixed neurons were incubated in preblock buffer (20 mM phosphate buffer, pH 7.4, 5% normal goat serum, 0.05% Triton X-100, and 450 mM NaCl) at 4° C. overnight with the following primary antibodies: mouse anti-CARM1 (1:200, Cell Signaling), rabbit anti-CARM1 (1:200, Cell Signaling), rabbit anti-NR2B (1:200, Millipore), mouse anti-NR2B (1:150, Thermo Fisher), mouse anti-PSD-95 (1:100, Cell Signaling), mouse anti-synapsin (1:200, Santa Cruz), or rabbit anti-synapsin (1:250, Millipore), followed by incubation with the appropriate secondary antibodies (Alexa 488-anti-rabbit or 546-anti-mouse; each at 1:1000, Invitrogen) in preblock buffer at RT for 1 hr. After immunostaining, the cells were mounted as previously described (50) and viewed on a Zeiss LSM 710 laser-scanning confocal microscope. Images were acquired under a 63× or 100× oil immersion objective lens and scanned for further analysis.

### Image Analysis

Confocal microscope images for each treatment from each of 3 independent sets of experiments were imported into Image J (NIH) software. To determine the proportion of CARM1 signal that co-localizes with PSD-95 or synapsin, Image J (NIH) software was used with Just Another Colocalization (JACoP) plugin set. A Mander's coefficient was calculated and the average value (N=10) was further analyzed. Dendritic spine length (from dendritic shaft to the top of spine head) and dendritic spine width (diameter of the widest part of spine head) were measured and dendritic spine density (number of spines/10 μm of dendrite) was also counted. Dendritic spine shape (stubby, mushroom, or filopodium) was identified as described in previous research (5, 21). After measuring spine length (the distance from the base of the neck to the furthest point on the spine heads) and diameter of spine head and neck, the ratio from each spine was calculated and compared. Thin spines were judged if serial viewing revealed the length to be greater than the neck diameter, and the diameters of the head and neck to be similar. Mushroom spines were judged if the diameter of the head was three times larger than the diameter of their necks. Stubby spines were judged if the diameter of the neck was similar to the total length of the spine.

### Quantification and Statistical Analysis

Quantitative data are expressed in arbitrary units (%) comparing untreated controls with cells treated with the indicated concentrations of inhibitors or transduced with siRNA. All data are presented as mean±SEM from 3 or more independent experiments unless otherwise indicated. Statistical comparisons between different treatment groups were conducted with Tukey's multiple comparison test after one-way ANOVA using GraphPad Prism 6 software (GraphPad Software Inc.). P values of less than 0.05 were considered to be statistically significant.

### Equivalents

The details of one or more embodiments of the invention are set forth in the accompanying description above. Although any methods and materials similar or equivalent to those described herein can be used in the practice or testing of the present invention, the preferred methods and materials are now described.

The foregoing description has been presented only for the purposes of illustration and is not intended to limit the invention to the precise form disclosed, but by the claims appended hereto.

### REFERENCES

1. Kandel, E. R., Dudai, Y., and Mayford, M. R. (2014) The molecular and systems biology of memory. Cell 157, 163-186
2. Sperling, R. A., Dickerson, B. C., Pihlajamaki, M., Vannini, P., LaViolette, P. S., Vitolo, O. V., Hedden, T., Becker, J. A., Rentz, D. M., Selkoe, D. J., and Johnson, K. A. (2010) Functional alterations in memory networks in early Alzheimer's disease. Neuromolecular Med. 12, 27-43
3. Walsh, D. M., and Selkoe, D. J. (2004) Deciphering the molecular basis of memory failure in Alzheimer's disease. Neuron 44, 181-193
4. Wondolowski, J., and Dickman, D. (2013) Emerging links between homeostatic synaptic plasticity and neurological disease. Front. Cell. Neurosci. 7, 223
5. Hongpaisan, J., Sun, M. K., and Alkon, D. L. (2011) PKCε activation prevents synaptic loss, Aβ elevation, and cognitive deficits in Alzheimer's disease transgenic mice. J. Neurosci. 31, 630-643
6. Harris, K. M., and Weinberg, R. J. (2012) Ultrastructure of synapses in the mammalian brain. Cold Spring Harb. Perspect. Biol. 4, a005587
7. Rochefort, N. L., and Konnerth, A. (2012) Dendritic spines: from structure to in vivo function. EMBO Rep. 13, 699-708
8. Amtul, Z., and Atta-Ur-Rahman (2015) Neural plasticity and memory: molecular mechanism. Rev. Neurosci. 26, 253-268
9. Koleske, A. J. (2013) Molecular mechanisms of dendrite stability. Nat. Rev. Neurosci. 14, 536-550
10. Kennedy, M. B. (2000) Signal-processing machines at the postsynaptic density. Science 290, 750-754
11. Newpher, T. M., and Ehlers, M. D. (2009) Spine microdomains for postsynaptic signaling and plasticity. Trends Cell Biol. 19, 218-227
12. Kastellakis, G., Cai, D. J., Mednick, S. C., Silva, A. J., and Poirazi, P. (2015) Synaptic clustering within dendrites: an emerging theory of memory formation. Prog. Neurobiol. 126, 19-35
13. Sala, C., and Segal, M. (2014) Dendritic spines: the locus of structural and functional plasticity. Physiol. Rev. 94, 141-188
14. Woolfrey, K. M., and Srivastava, D. P. (2016) Control of Dendritic Spine Morphological and Functional Plasticity by Small GTPases. Neural Plast. 2016, 3025948
15. Saneyoshi, T., Fortin, D. A., and Soderling, T. R. (2010) Regulation of spine and synapse formation by activity-dependent intracellular signaling pathways. Curr. Opin. Neurobiol. 20, 108-115
16. Nelson, T. J., Sun, M. K., Hongpaisan, J., and Alkon, D. L. (2008) Insulin, PKC signaling pathways and synaptic remodeling during memory storage and neuronal repair. Eur. J. Pharmacol. 585, 76-87
17. Sun, M. K., and Alkon, D. L. (2010) Pharmacology of protein kinase C activators: cognition-enhancing and antidementic therapeutics. Pharmacol. Ther. 127, 66-77
18. Calabress, B., and Halpain, S. (2005) Essential role for the PKC target MARCKS in maintaining dendritic spine morphology. Neuron 48, 77-90
19. Matsuoka, Y., Li, X., and Bennett, V. (1998) Adducin is an in vivo substrate for protein kinase C: phosphorylation in the MARCKS-related domain inhibits activity in promoting spectrin-actin complexes and occurs in many cells, including dendritic spines of neurons. J. Cell Biol. 142, 485-497
20. Pascale, A., Amadio, M., Scapagnini, G., Lanni, C., Racchi, M., Provenzani, A., Govoni, S., Alkon, D. L., and Quattrone, A. (2005) Neuronal ELAV proteins enhance mRNA stability by a PKCalpha-dependent pathway. Proc. Natl. Acad. Sci. U.S.A. 102, 12065-12070
21. Hongpaisan, J., and Alkon D. L. (2007) A structural basis for enhancement of long-term associative memory in single dendritic spines regulated by PKC. Proc. Natl. Acad. Sci. U.S.A. 104, 19571-19576
22. Lim, C. S., and Alkon, D. L. (2012) Protein kinase C stimulates HuD-mediated mRNA stability and protein expression of neurotrophic factors and enhances dendritic maturation of hippocampal neurons in culture. Hippocampus 22, 2303-2319
23. Guo, A., Gu, H., Zhou, J., Mulhern, D., Wang, Y., Lee, K. A., Yang, V., Aguiar, M., Kornhauser, J., Jia, X., Ren, J., Beausoleil, S. A., Silva, J. C., Vemulapalli, V., Bedford, M. T., and Comb, M. J. (2014) Immunoaffinity enrichment and mass spectrometry analysis of protein methylation. Mol. Cell. Proteomics 13, 372-387
24. Morales, Y., Caceres, T., May, K., and Hevel, J. M. (2016) Biochemistry and regulation of the protein arginine methyltransferases (PRMTs). Arch. Biochem. Biophys. 590, 138-152
25. Wolf, S. S. (2009) The protein arginine methyltransferase family: an update about function, new perspectives and the physiological role in humans. Cell. Mol. Life Sci. 66, 2109-2121
26. Hu, H., Qian, K., Ho, M. C., and Zheng, Y. G. (2016) Small Molecule Inhibitors of Protein Arginine methyltransferases. Expert Opin. Investig. Drugs 25, 335-358
27. McBride, A. E. (2006) 3 Diverse roles of protein arginine methyltransferases. Enzymes 24, 51-103
28. Colombrita, C., Silani, V., and Ratti, A. (2013) ELAV proteins along evolution: back to the nucleus? Mol. Cell. Neurosci. 56, 447-455
29. Fujiwara, T., Mori, Y., Chu, D. L., Koyama, Y., Miyata, S., Tanaka, H., Yachi, K., Kubo, T., Yoshikawa, H., and Tohyama, M. (2006) CARM1 regulates proliferation of PC12 cells by methylating HuD. Mol. Cell. Biol. 26, 2273-2285
30. Higashimoto, K., Kuhn, P., Desai, D., Cheng, X., and Xu, W. (2007) Phosphorylation-mediated inactivation of coactivator-associated arginine methyltransferase 1. Proc. Natl. Acad. Sci. U.S.A. 104, 12318-12323
31. Feng, Q., He, B., Jung, S. Y., Song, Y., Qin, J., Tsai, S. Y., Tsai, M. J., and O'Malley, B. W. (2009) Biochemical control of CARM1 enzymatic activity by phosphorylation. J. Biol. Chem. 284, 36167-36174
32. Zhang, X., and Cheng, X. (2003) Structure of the predominant protein arginine methyltransferase PRMT1 and analysis of its binding to substrate peptides. Structure 11, 509-520
33. Nimchinsky, E. A., Sabatini, B. L., and Svoboda, K. (2002) Structure and function of dendritic spines. Annu. Rev. Physiol. 64, 313-353
34. Hunt, D. L., and Castillo, P. E. (2012) Synaptic plasticity of NMDA receptors: mechanisms and functional implications. Curr. Opin. Neurobiol. 22, 496-508
35. Shepherd, J. D., and Huganir, R. L. (2007) The cell biology of synaptic plasticity: AMPA receptor trafficking. Annu. Rev. Cell Dev. Biol. 23, 613-643
36. Cunha, C., Brambilla, R., and Thomas, K. L. (2010) A simple role for BDNF in learning and memory? Front. Mol. Neurosci. 3, 1-14
37. Shen, K., and Cowan, C. W. (2010) Guidance molecules in synapse formation and plasticity. Cold Spring Harb. Perspect. Biol. 2, a001842
38. Waterhouse, E. G., and Xu, B. (2009) New insights into the role of brain-derived neurotrophic factor in synaptic plasticity. Mol. Cell. Neurosci. 42, 81-89
39. Bedford, M. T. (2007) Arginine methylation at a glance. J. Cell Sci. 120, 4243-4246
40. Lee, Y. H., and Stallcupt, M. R. (2009) Minireview: protein arginine methylation of nonhistone proteins in transcriptional regulation. Mol. Endocrinol. 23, 425-433
41. Yuste, R., and Bonhoeffer, T. (2001) Morphological changes in dendritic spines associated with long-term synaptic plasticity. Annu. Rev. Neurosci. 24, 1071-1089
42. Schubert, V., and Dotti, C. G. (2007) Transmitting on actin: synaptic control of dendritic architecture. J. Cell Sci. 120, 205-212
43. Carlisle, H. J., and Kennedy, M. B. (2005) Spine architecture and synaptic plasticity. Trends Neurosci. 28, 182-187
44. Tashiro, A., and Yuste, R. (2004) Regulation of dendritic spine motility and stability by Racl and Rho kinase: evidence for two forms of spine motility. Mol. Cell. Neurosci. 26, 429-440
45. Pontrello, C. G., and Ethell, I. M. (2009) Accelerators, Brakes, and Gears of Actin Dynamics in Dendritic Spines. Open Neurosci. J. 3, 67-86
46. Zhu, J., Shang, Y., and Zhang, M. (2016) Mechanistic basis of MAGUK-organized complexes in synaptic development and signalling. Nat. Rev. Neurosci. 17, 209-223
47. Nagura, H., Ishikawa, Y., Kobayashi, K., Takao, K., Tanaka, T., Nishikawa, K., Tamura, H., Shiosaka, S., Suzuki, H., Miyakawa, T., Fujiyoshi, Y., and Doi, T. (2012) Impaired synaptic clustering of postsynaptic density proteins and altered signal transmission in hippocampal neurons, and disrupted learning behavior in PDZ1 and PDZ2 ligand binding-deficient PSD-95 knockin mice. Mol. Brain 5, 43
48. Basso, M., and Pennuto, M. (2015) Serine phosphorylation and arginine methylation at the crossroads to neurodegeneration. Exp. Neurol. 271, 77-83
49. Routtenberg, A., and Rekart, J. L. (2005) Post-translational protein modification as the substrate for long-lasting memory. Trends Neurosci. 28, 12-19
50. Walikonis, R. S., Jensen, O. N., Mann, M., Provance, D. W. Jr., Mercer, J. A., and Kennedy, M. B. (2000) Identification of proteins in the postsynaptic density fraction by mass spectrometry. J. Neurosci. 20, 4069-4080
51. Lim, C. S., and Walikonis, R. S. (2008) Hepatocyte growth factor and c-Met promote dendritic maturation during hippocampal neuron differentiation via the Akt pathway. Cell. Signal. 20, 825-835

1.-53. (canceled)

54. A method of promoting regenerative synaptogenesis in a patient comprising administering a pharmaceutically effective amount of an inhibitor of an arginine methyltransferase to the patient, wherein the patient is suffering from a disorder characterized by inadequate or deficient synaptogenesis.

55. The method of claim 54, wherein the arginine methyltransferase is coactivator-associated arginine methyltransferase 1 (CARM1).

56. The method of claim 54, wherein the inhibitor of an arginine methyltransferase is selected from the group consisting of S-(5′-Adenosyl)-L-homocysteine; 7,7′-(carbonylbis(azanediyl)bis(4-oxidonaphthalene-2-sulfonate); 5′-deoxy-5′(methylthio)adenosine;

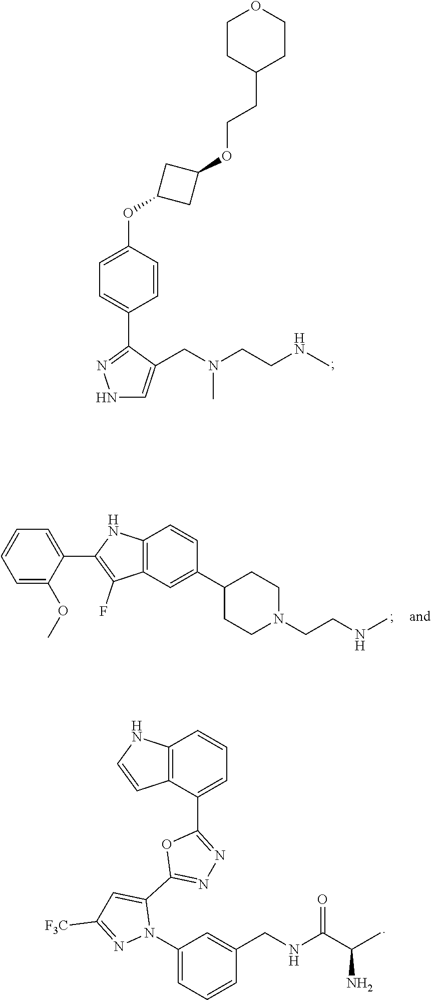 [SMILES: CNCCN(C)Cc1c[nH]nc1-c1ccc(O[C@H]2C[C@H](OCCC3CCOCC3)C2)cc1 ; CNCCN1CCC(c2ccc3[nH]c(-c4ccccc4OC)c(F)c3c2)CC1 ; C[C@@H](N)C(=O)NCc1cccc(-n2nc(C(F)(F)F)cc2-c2nnc(-c3cccc4[nH]ccc34)o2)c1]

57. The method of claim 54, wherein the disorder is Alzheimer's disease, Parkinson's disease, Amyotrophic lateral sclerosis, or Huntington's disease.

58. The method of claim 54, wherein the pharmaceutically effective amount of the inhibitor of an arginine methyltransferase is from about 0.0000001 mg/kg to about 250 mg/kg per dose.

59. The method of claim 54, wherein the pharmaceutically effective amount of the inhibitor of an arginine methyltransferase is from about 0.0001 mg/kg to about 5 mg/kg per dose.

60. The method of claim 54 wherein the inhibitor of an arginine methyltransferase is administered in combination with a PKC activator.

61. The method of claim 54 wherein the inhibitor of an arginine methyltransferase is administered in combination with bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog or any combination thereof.

62. The method of claim 54 wherein the inhibitor of an arginine methyltransferase is administered in combination with bryostatin 1.

63. The method of claim 54 wherein the inhibitor of an arginine methyltransferase is administered in combination with a polyunsaturated fatty acid, a potassium channel activator, a neristatin, phorbol-12-myristate-13-acetate (PMA), okadaic acid, 1α,25-dihydroxyvitamin D3, 12-deoxyphorbol-13-acetate (prostratin), 1,2-dioctanoyl-sn-glycerol (DOG), 1-oleoyl-2-acetyl-sn-glycerol (OAG), (2S,5 S)-(E,E)-8-(5-(4-(trifluoromethyl)phenyl)-2,4-pentadienoylamino)benzolactam (α-amyloid precursor protein modulator), cis-9-octadecenoic acid (oleic acid), ingenol 3-angelate, resiniferatoxin, L-α-Phosphatidyl-D-myo-inositol-4,5-bisphosphate, triammonium salt (PIP2), phorbol-12, 13-dibutyrate, 8(S-hydroxy-(5Z, 9E, 11Z, 14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12, 13-dihexanoate, prostratin and its analogs, resiniferonol 9,13,14-ortho-phenylacetate, C-8 ceramide, 1,6-bis(Cyclohexyloximinocarbonylamino) hexane;
1,6-Di(O-(carbamoyl)cyclohexanone oxime) hexane (RHC-80267), (+/−)-1-oleoyl-2-acetylglycerol, 5(S),6(R), 1 5(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4a-phorbol 12, 13-dibutyrate, 1,2-dihexanoyl-sn-glycerol, zoledronic acid disodium salt tetrahydrate, arachidonic acid methyl ester, diazoxide, neristatin 1, or arachidonic acid-d8.
